(19) United States
(12) Patent Application Publication
(10) Pub. No.: US 2007/0299136 A1
(43) Pub. Date: Dec. 27, 2007

(54) Crystalline and Amorphous Forms of Efaproxiral Sodium

(75) Inventors: Douglas G. Johnson, Arvada, CO (US); Chris Murray, Arlington, MA (US); Stephan D. Parent, West Lafayette, IN (US); David T. Jonaitis, Lafayette, IN (US)

Correspondence Address: SWANSON & BRATSCHUN, L.L.C., 8210 SOUTHPARK TERRACE, LITTLETON, CO, 80120, US

(73) Assignee: ALLOS THERAPEUTICS, INC., Westminster, CO (US)

(21) Appl. No.: 10/598,854

(22) PCT Filed: Apr. 22, 2005
(86) PCT No.: PCT/US05/13709

§ 371 Date: May 15, 2007

Related U.S. Application Data
(60) Provisional application No. 60/564,308, filed on Apr. 22, 2004.
(60) Provisional application No. 60/564,721, filed on Apr. 22, 2004.

(51) Int. Cl. A61K 31/196; A61P 11/00; A61P 43/00; A61P 9/02; A61P 9/10; C07C 63/00

(52) U.S. Cl. 514/563; 562/405

(57) ABSTRACT

There are provided in accordance with the present invention crystalline forms A, B, C, F, G, I, J, P, and Q of efaproxiral sodium. Also provided is the amorphous form of efaproxiral sodium. Also provided are methods of forming the novel polymorphs and the amorphous form of efaproxiral sodium, therapeutic methods utilizing them and pharmaceutical dosage forms containing them.

This application claims priority to U.S. Provisional Patent Application Ser. No. 60/564,721, filed Apr. 22, 2004, entitled “Compositions of Allosteric Hemoglobin Modifiers and Methods of Making the Same,” and to U.S. Provisional Patent Application Ser. No. 60/564,308, filed Apr. 22, 2004, entitled “Crystalline Forms of RSR13 Sodium Salt,” each of which is incorporated herein by reference in their entirety.

### FIELD OF THE INVENTION

This disclosure relates to the isolation of crystalline polymorphic forms of efaproxiral sodium and the amorphous form of efaproxiral sodium, as well as crystalline forms of certain solvates of efaproxiral sodium in particular solvates where the solvent portion of the lattice structure may be water, an alcohol (such as ethanol and/or methanol), or acetone. Efaproxiral sodium is also known as 2-[4-[2-[(3,5-dimethylphenyl)amino]-2-oxoethyl]phenoxy]-2-methyl-propionic acid, sodium salt; also known as RSR13 sodium salt. Efaproxiral sodium is used in the treatment of disease, including the treatment of cancers.

### BACKGROUND

The polymorphic behavior of drugs can be of crucial importance in pharmacy and pharmacology. Polymorphs are, by definition, different crystal packing arrangements of the same molecule. These different packing arrangements of the molecule in the crystal lattice often lead to different physical properties. When a solvent molecule(s) is contained within the crystal lattice the resulting crystal is called a solvate. Solvates are sometimes known as pseudopolymorphs. Crystalline solvates are also characterized by unique crystal packing arrangements giving rise to their own polymorphs. If the solvent molecule(s) within the crystal structure is a water molecule, then the solvate (pseudopolymorph) is called a hydrate. The differences in physical properties exhibited by polymorphs or solvates affect pharmaceutical parameters such as storage stability, compressibility and density (important in formulation and product manufacturing), and dissolution rates (an important factor in determining bio-availability). (See H. Brittain, Polymorphism in Pharmaceutical Solids, Marcel Dekker, New York, N.Y., 1999, pp. 1-2). Differences in stability result from changes in chemical reactivity (e.g. differential oxidation, such that a dosage form discolors more rapidly when comprised of one polymorph than when comprised of another polymorph) or mechanical changes (e.g. tablets crumble on storage as a kinetically favored polymorph converts to thermodynamically more stable polymorph) or both (e.g. tablets of one polymorph are more susceptible to breakdown at high humidity). As a result of solubility/dissolution differences, in the extreme case, some polymorphic transitions may result in lack of potency and/or decreased bio-availability, or, at the other extreme, toxicity. In addition, the physical properties of the crystal may be important in processing: for example, one polymorph might be more likely to form solvates or might be difficult to filter and wash free of impurities (i.e particle shape and size distribution might be different between one polymorph relative to the other).

Polymorphic and pseudopolymorphic forms of the drug substance (also known as the “active pharmaceutical ingredient” (API)), as administered by itself or formulated as a drug product (also known as the final or finished dosage form, or as the pharmaceutical composition) are well known and affect, for example, the solubility, stability, flowability, fractability, and compressibility of drug substances and the safety and efficacy of drug products, (see, e.g., Knapman, K Modern Drug Discoveries, March 2000: 53).

The preparation and uses for 2-[4-[2-[(3,5-dimethylphenyl)amino]-2-oxoethyl]phenoxy]-2-methyl-propionic acid (also known as efaproxiral and RSR13) and its physiologically acceptable salts have been described previously in U.S. Pat. Nos. 5,049,695; 5,122,539; 5,290,803; 5,432,191; 5,525,630; 5,648,375; 5,661,182; 5,677,330; 5,705,521; 5,872,888; and 5,927,283, and U.S. Patent Application Publication No. 20030017612 A1, each of which is incorporated herein by reference.

### SUMMARY

The disclosure provides novel crystalline forms of efaproxiral sodium, hereinafter referred to as Forms A, B, C, F, G, I, J, P, and Q, and also provides an amorphous form of efaproxiral sodium.

In one embodiment, the disclosure provides the unsolvated crystalline efaproxiral sodium forms termed Form A and B.

In another embodiment, the disclosure provides crystalline solvates of efaproxiral sodium comprising efaproxiral sodium and a solvent selected from group consisting of water, ethanol, methanol and acetone.

In one embodiment, the disclosure provides the crystalline hydrates of efaproxiral sodium termed Form C, Form J and Form I.

In another embodiment, the disclosure provides the crystalline ethanolates of efaproxiral sodium termed Form P and Form G.

In another embodiment, the disclosure provides the crystalline acetone solvate of efaproxiral sodium termed Form Q.

In another embodiment, the disclosure provides the crystalline methanolate of efaproxiral sodium termed Form F.

In another embodiment, the disclosure provides amorphous efaproxiral sodium.

In another embodiment, the disclosure provides a pharmaceutical formulation comprising any of the aforementioned crystalline forms of efaproxiral sodium or the amorphous form of efaproxiral sodium and one or more pharmaceutical carriers, diluents, or excipients.

In another embodiment, the disclosure provides a method for the preparation of an aqueous solution of efaproxiral sodium, the method comprising dissolving any of the aforementioned crystalline forms of efaproxiral sodium or the amorphous form of efaproxiral sodium in a solution comprising water.

In another embodiment, the disclosure provides aqueous solutions of efaproxiral sodium produced by dissolving any of the aforementioned crystalline forms of efaproxiral sodium or the amorphous form of efaproxiral sodium in a solution comprising water.

In another embodiment, the disclosure provides methods of preparing each of the aforementioned crystalline forms of efaproxiral sodium and the amorphous form of efaproxiral sodium.

In another embodiment, the disclosure provides a method for treating a condition selected from the group consisting of whole body or tissue hypothermia, hypoxia or hypotension, wounds, brain injury, diabetic ulcers, chronic leg ulcers, pressure sores, tissue transplants, stroke or cerebro ischemia, ischemia or oxygen deprivation, respiratory disorders including acute respiratory distress syndrome and chronic obstructive pulmonary disorder, surgical blood loss, sepsis, multi-system organ failure, normovolemic hemodilution procedures, carbon monoxide poisoning, bypass surgery, carcinogenic tumors, and oxygen deprivation of a fetus comprising the step of administering to a patient suffering from or undergoing said condition a sufficient quantity of any of the aforementioned crystalline forms of efaproxiral sodium, the amorphous form of efaproxiral sodium, any of the aforementioned pharmaceutical formulations, or any of the aforementioned aqueous solutions of efaproxiral sodium.

### BRIEF DESCRIPTION OF THE FIGURES

FIG. 1 depicts observed interconversions between the crystalline and amorphous forms of efaproxiral sodium.

FIG. 2 depicts the X-Ray Powder Diffraction (XRPD) pattern of Form A efaproxiral sodium.

FIG. 3 depicts the Fourier transform infrared (FTIR) spectrum of Form A efaproxiral sodium.

FIG. 4 depicts the XPRD pattern of Form B efaproxiral sodium.

FIG. 5 depicts the FTIR spectrum of Form B efaproxiral sodium.

FIG. 6 depicts an ORTEP drawing of Form A efaproxiral sodium (atoms are represented by 50% probability anisotropic thermal ellipsoids). The asymmetric unit shown contains six efaproxiral molecules coordinating to six sodium cations.

FIG. 7 depicts the proposed structure of efaproxiral sodium.

FIG. 8 depicts the XRPD pattern of Form I efaproxiral sodium.

FIG. 9 depicts the FTIR spectrum of Form I efaproxiral sodium.

FIG. 10 depicts a plot of weight change % (and equivalent number of moles of H₂O) of a sample of efaproxiral sodium (starting as Form A) versus relative humidity as the humidity of the environment surrounding the sample of efaproxiral sodium is first raised from less than 5% to about 95% (open circles indicates adsorption trace) and then the humidity is decreased back down to about 5% (closed circles indicate desorption trace).

FIG. 11 depicts the XRPD pattern of Form J efaproxiral sodium.

FIG. 12 depicts the FTIR spectrum of Form J efaproxiral sodium.

FIG. 13 depicts the XRPD pattern of Form C efaproxiral sodium.

FIG. 14 depicts the FTIR spectrum of Form C efaproxiral sodium.

FIG. 15 depicts the XRPD pattern of Form P efaproxiral sodium.

FIG. 16 depicts the FTIR spectrum of Form P efaproxiral sodium.

FIG. 17 depicts the percentage weight lost by a sample of Form P efaproxiral sodium as the temperature is raised from ambient to 165° C.

FIG. 18 indicates that the FTIR spectrum of the volatile lost (top trace) from Form P efaproxiral sodium as the temperature is raised from ambient to 165° C. is the same as the FTIR spectrum of ethanol (bottom trace).

FIG. 19 depicts the XRPD pattern of Form P efaproxiral sodium prior to heating (top trace) and the XRPD pattern of the solid material remaining after Form P efaproxiral sodium is heated to 165° C.

FIG. 20 depicts the XRPD pattern of Form G efaproxiral sodium.

FIG. 21 depicts the XRPD pattern of Form Q efaproxiral sodium.

FIG. 22 depicts the FTIR spectrum of Form Q efaproxiral sodium.

FIG. 23 depicts the percentage weight lost by a sample of Form Q efaproxiral sodium as the temperature is raised from ambient to 165° C.

FIG. 24 indicates that the FTIR spectrum of the volatile lost (top trace) from Form Q efaproxiral sodium as the temperature is raised from ambient to 165° C. is the same as the FTIR spectrum of acetone (bottom trace).

FIG. 25 depicts the XRPD pattern of Form Q efaproxiral sodium prior to heating (top trace) and the XRPD pattern of the solid material remaining after Form Q efaproxiral sodium is heated to 165° C.

FIG. 26 depicts the XRPD of Form F efaproxiral sodium.

FIG. 27 depicts the FTIR spectrum of Form F efaproxiral sodium.

FIG. 28 depicts an ORTEP drawing of Form F efaproxiral sodium (atoms are represented by 50% probability anisotropic thermal ellipsoids). The asymmetric unit shown contains six efaproxiral and methanol molecules coordinated to six sodium cations.

FIG. 29 depicts the XRPD pattern of amorphous efaproxiral sodium.

### DETAILED DESCRIPTION OF THE PREFERRED EMBODIMENTS

Efaproxiral (X=H⁺): 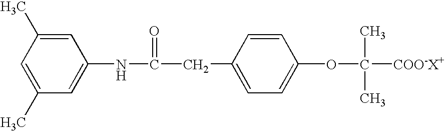
is an allosteric effector of hemoglobin, and has been shown to enhance tissue oxygenation in vivo. Efaproxiral (when X=H⁺) is represented by the names 2-[4-(((3,5-dimethylanilino)carbonyl)methyl)phenoxy]-2-methylpropionic acid) or 2-[4-[2-[(3,5-dimethylphenyl)amino]-2-oxoethyl]phenoxy]-2-methylpropanoic acid. In general, the drug product of efaproxiral is prepared from a physiologically acceptable salt, such as the monosodium salt; that is, X⁺ is Na⁺. Efaproxiral induces allosteric modification of hemoglobin, such that its binding affinity for oxygen is decreased, resulting in increased oxygen distribution to tissues by erythrocytes. The sodium salt of efaproxiral is hereinafter referred to as efaproxiral sodium. A method for the preparation of purified efaproxiral sodium and precursors thereof is provided in EXAMPLE 1 (see also U.S. Provisional Patent Application Ser. No. 60/60/564,721, filed Apr. 22, 2004 incorporated herein by reference in its entirety).

The ability to allosterically modify hemoglobin also allows the compounds to be useful in treating a variety of disorders and conditions mediated through allosterically modifying hemoglobin to shift oxygen equilibrium in favor of free oxygen. Such disorders may include, but are not limited to, whole body or tissue hypothermia, hypoxia or hypotension, wounds, brain injury, diabetic ulcers, chronic leg ulcers, pressure sores, tissue transplants, stroke or cerebro ischemia, ischemia or oxygen deprivation, respiratory disorders including acute respiratory distress syndrome and chronic obstructive pulmonary disorder, surgical blood loss, sepsis, multi-system organ failure, normovolemic hemodilution procedures, carbon monoxide poisoning, bypass surgery, carcinogenic tumors, oxygen deprivation of a fetus. The compound is useful in a method comprising the step of administering to a patient suffering from or undergoing the claimed condition a sufficient quantity of an allosteric effector compound. In the case of carcinogenic tumors, the compounds are useful alone, and as radiosensitizers in conjunction with ionizing radiation (See Teicher, (1996) Drug Dev. Res. 38:1-11; Rockwell and Kelley (1998) Rad. Oncol. Invest. 6:199-208; and Khandelwal et al. (1996) Rad. Oncol. Invest. 4:51-59). The allosteric modification properties also allow it to be useful in certain imaging methods, especially blood oxygen level dependent MRI, and also in diagnostic methods, including determination of tumor oxygenation, and determination of an optimal time for commencing radiation treatment based on tumor oxygenation. The preparation and uses for efaproxiral and its physiologically acceptable salts has been described previously in U.S. Pat. Nos. 5,049,695; 5,122,539; 5,290,803; 5,432,191; 5,525,630; 5,648,375; 5,661,182; 5,677,330; 5,705,521; 5,872,888; and 5,927,283, and U.S. Patent Application Publication No. 20030017612 A1. These patents also describe the preparation and use of structurally similar compounds. Other patents describing closely related compounds include U.S. Pat. No. 5,248,785; 5,731,454. These patents, applications, and all other patents, applications, and publications referred to herein, are specifically incorporated by reference herein.

A screen for polymorphs and solvates of efaproxiral sodium was performed using a variety of crystallization techniques. The resulting polymorphs were analyzed using a number of analytical techniques well known in the art for their ability to differentiate between different polymorphs, including thermogravimetric analysis (TGA), reflectance Fourier transform infrared (FTIR) spectroscopy (see EXAMPLE 2), and X-ray powder diffraction (XRPD) (see EXAMPLE 3). TGA is often very useful for distinguishing between different solid forms of a material because the temperature(s) at which a physical change in a material occurs is usually characteristic of the polymorph or solvate. X-Ray Powder Diffraction (XRPD) is a technique that detects long-range and short-range order in a crystalline material. IR spectroscopy also detects both intramolecular and intermolecular bonding in solids, and further provides information regarding the chemical composition of the crystalline material.

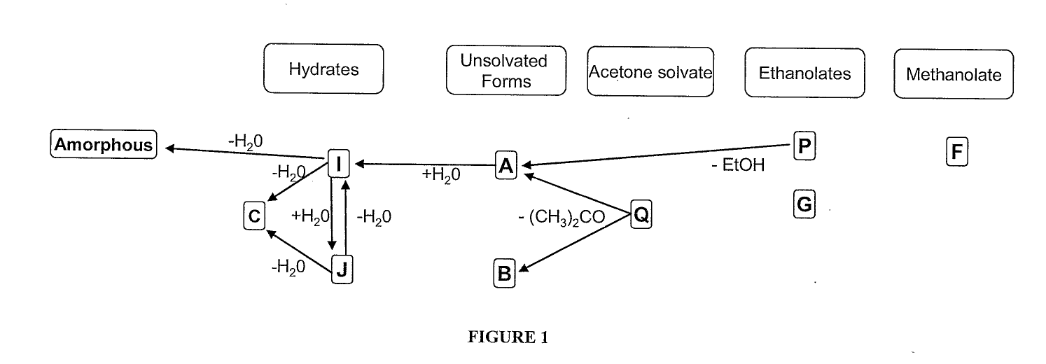

The disclosure provides nine novel polymorphs of efaproxiral sodium, hereinafter referred to as Forms A, B, C, F, G, I, J, P, and Q. Forms C, F, G, I, J, P, and Q are crystalline solvates of efaproxiral sodium and Forms A and B are unsolvated. Based on the single crystal structures obtained for Form A and Form F, it is believed that the crystal lattice of efaproxiral sodium is characterized by sodium channels around which the efaproxiral molecules pack. Without being bound by theory, it is believed that varying amounts of solvent molecules can be present in these channels without destroying the crystalline form. Conversions between the polymorphs that have been observed are depicted schematically in FIG. 1. This is not to indicate a limit on the ways or paths that could be used but rather represents examples that have been observed.

The XRPD patterns of each of Forms A, B, C, F, G, I, J, P, and Q feature sharp peaks indicative of highly crystalline materials. Although many of the intense reflections observed for the polymorphs are generally at similar diffraction angles, each of the forms gives a different powder pattern, allowing for a clear distinction between the individual polymorphs. It is well known in the crystallography art that, for any given polymorph, the relative intensities of the diffraction peaks may vary due to preferred orientation resulting from factors such as crystal morphology, sample preparation, or due to other effects. Where the effects of preferred orientation are present, peak intensities are altered, but the characteristic peak positions of the polymorph are unchanged. See, e.g., The United States Pharmacopeia #23, National Formulary #18, pages 1843-1844, 1995.

The disclosure also provides amorphous efaproxiral sodium. The XRPD of amorphous efaproxiral sodium lacks sharp reflectance peaks, which is typical for amorphous solids.

The aforementioned crystalline forms and the amorphous form of efaproxiral sodium may be used to formulate pharmaceutical compositions. The resulting pharmaceutical compositions may be administered to patients in order to treat a variety of disorders and conditions by allosterically modifying hemoglobin to shift oxygen equilibrium in favor of free oxygen, as discussed above. In the case of carcinogenic tumors, the compounds and pharmaceutical compositions provided herein are useful alone, and also as radiosensitizers in conjunction with ionizing radiation.

The discovery of new polymorphic forms and the amorphous form of efaproxiral sodium provide an opportunity to improve the performance characteristics of a pharmaceutical product comprising efaproxiral sodium as the API. It enlarges the repertoire of materials that a formulation scientist has available for designing, for example, a pharmaceutical dosage form of efaproxiral sodium with a targeted release profile or other desired characteristic. Knowing the rate of dissolution of all the crystalline forms and the amorphous form is useful in the preparation of drug solutions. It is clearly advantageous when this repertoire is enlarged by the discovery of new solvated crystalline forms of efaproxiral sodium.

The new polymorphic forms and the amorphous form of efaproxiral sodium disclosed herein may possess different physical properties including, for example, the flowability of the milled solid. Flowability affects the ease with which the material is handled during processing into efaproxiral sodium. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important physical property of the new crystal forms and the amorphous form of efaproxiral sodium relate to its rate of dissolution in aqueous fluid. The rate of dissolution of an active ingredient in a patient's stomach fluid can have therapeutic consequences since it imposes an upper limit on the rate at which an orally-administered efaproxiral sodium can reach the patient's bloodstream. The rate of dissolution is also a consideration in formulating solutions, syrups, elixirs and other liquid medicaments. The solid state form of efaproxiral sodium may also affect its behavior on compaction and its storage stability—both in bulk and once formulated.

In the description that follows (including the section entitled “EXAMPLES”) all specified quantities and process conditions (including time, temperature, and the like) are examples only and are understood to include a range of equivalents. All such numerical examples are understood to be modified by the term “about,” whether or not this is explicitly stated, and the scope of the term “about” is a range of values as could be determined by one of ordinary skill in the art without undue experimentation.

In the description that follows, all angular XRPD peak positions in 20 are obtained from a copper radiation source ((CuKα λ=1.54 Å). All IR absorption bands are obtained from reflectance Fourier transform infrared (FTIR) spectroscopy.

Unsolvated Forms of Efaproxiral Sodium

Form A is a new polymorph of efaproxiral sodium that may be obtained by recrystallizing efaproxiral sodium from ethanol and acetone. For example, Form A (colorless needles) may be obtained by: dissolving efaproxiral sodium in water to form an aqueous solution; then concentrating the aqueous solution to remove the maximum amount of water while maintaining the aqueous solution at a temperature of about 50° C.; then adding ethanol to the concentrated aqueous solution to provide a mixture having less than 15 weight percent water content; then cooling the ethanol/water mixture without precipitating the efaproxiral sodium from the ethanol/water mixture; then adding acetone to the ethanol/water mixture to precipitate crystalline efaproxiral sodium; and then cooling the mixture to below about 25° C. with stirring (see EXAMPLE 4).

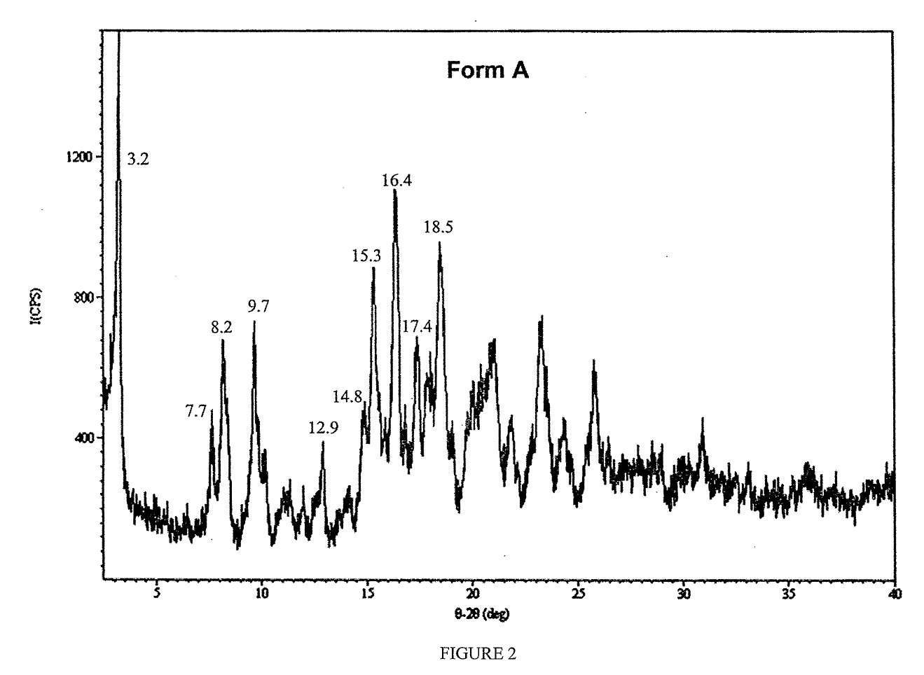

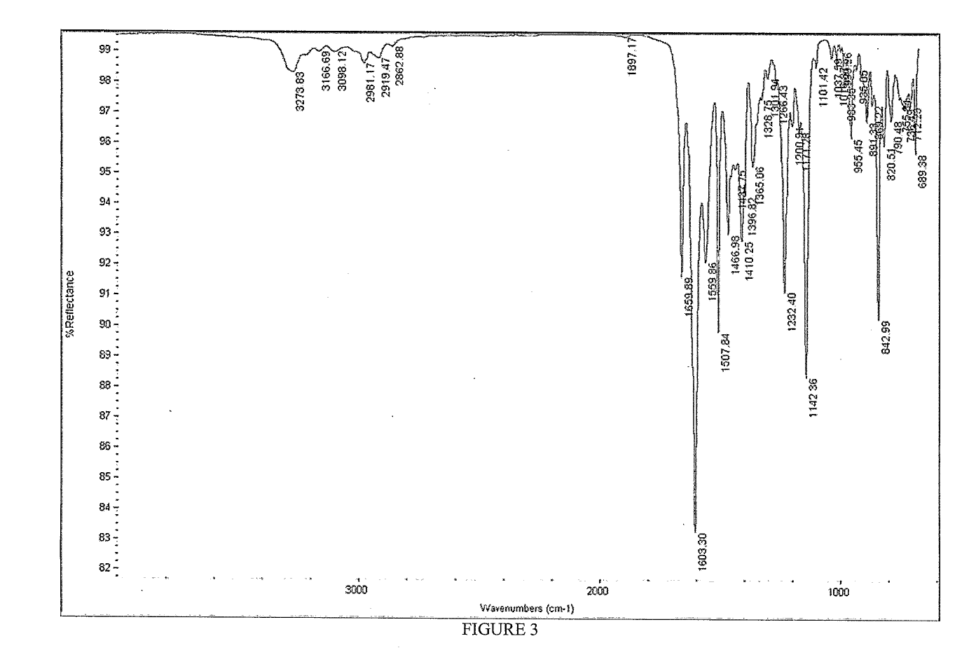

The XRPD pattern of Form A is presented in FIG. 2, and the FTIR spectrum of Form A is presented in FIG. 3. Based on extensive drying experiments, Form A appears to be unsolvated.

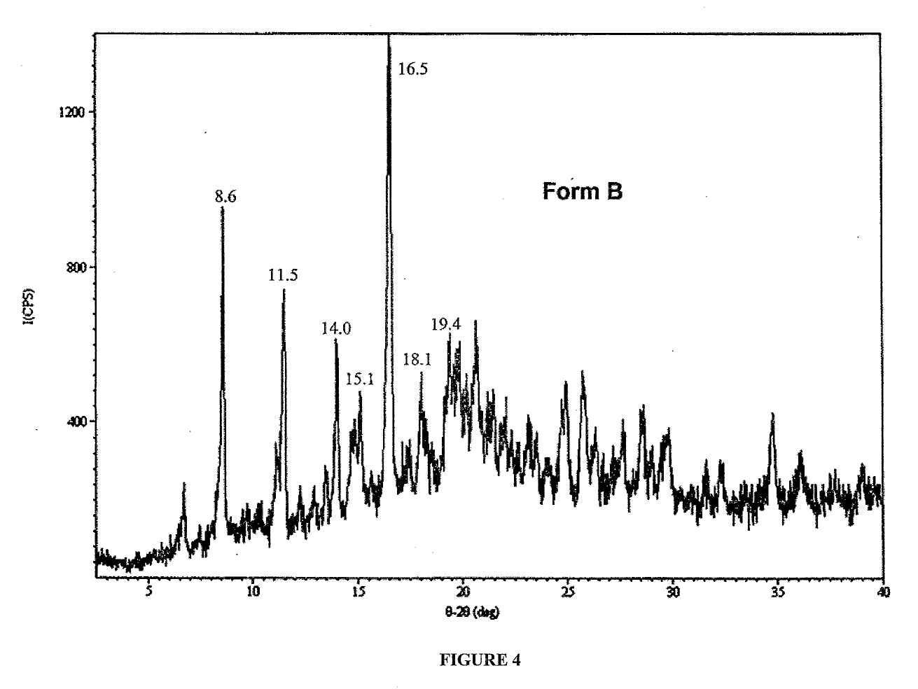

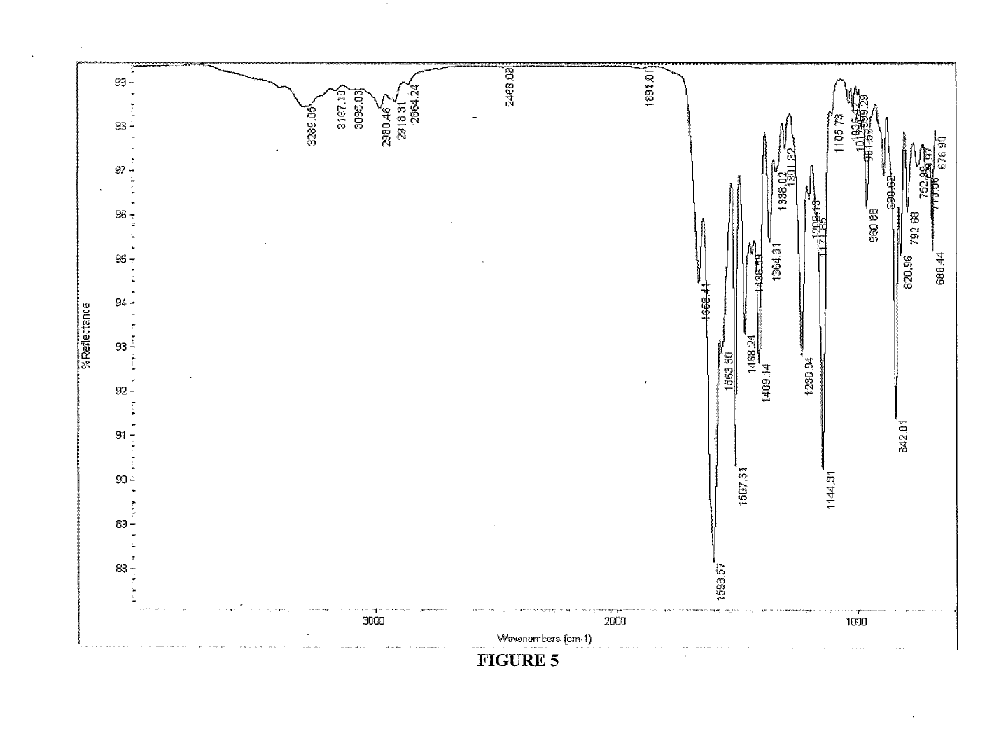

Form B is a new polymorph of efaproxiral sodium that can be formed by dissolving efaproxiral sodium in acetone and water to form a solution, then cooling the solution to precipitate the efaproxiral sodium crystals. See EXAMPLE 5. The XRPD pattern of Form B is presented in FIG. 4, and the FTIR spectrum of Form B is presented in FIG. 5. Based on extensive drying experiments, Form B appears to be unsolvated.

Table 1 below tabulates certain angular XRPD peak positions in 20 from Form A (FIG. 2) and Form B (FIG. 4) when obtained from a copper radiation source (CuKα λ=1.54 Å).

TABLE 1

| Form A | Form B |
|---|---|
| 2θ (degrees) | 2θ (degrees) |
| 3.23 | 8.59 |
| 7.65 | 11.46 |
| 8.2 | 13.98 |
| 9.66 | 15.1 |
| 12.86 | 16.54 |
| 14.8 | 18.01 |
| 15.31 | 19.36 |
| 16.38 | 20.61 |
| 17.37 | 24.74 |
| 18.51 | 24.92 |
| 23.26 | 25.75 |
| 25.77 | |

Based on peak positions, and to some extent on peak intensities also, Form A possesses characteristic XRPD peaks at 3.2±0.2° and 9.7±0.2° in 2θ in contrast to the XRPD of Form B. Form B possesses characteristic XRPD peaks 11.5±0.2°, 14.0±0.2°, and 19.4±0.2° in 2θ in contrast to the XRPD pattern of Form A. Hence, it is possible to differentiate between the unsolvated forms of efaproxiral sodium using at least the aforementioned characteristic peaks, or a combination of the aforementioned characteristic peaks and the other peaks in Table 1.

Alternatively, Form A may also be identified by a unique reflectance Fourier transform infrared (FTIR) absorption band at 3274±2 cm⁻¹, or by a unique FTIR absorption band at 955±2 cm⁻¹, or by a unique FTIR absorption band at 736±2 cm⁻¹, or by any combination of these unique FTIR absorption bands. Form B may also be identified by a unique FTIR absorption band at 3289±2 cm⁻¹; or by a unique FTIR absorption band at 1338±2 cm⁻¹, or by a unique FTIR absorption band at 730±2 cm⁻¹; or by any combination of these unique FTIR absorption bands.

The crystal structure of Form A efaproxiral sodium was determined using single crystal X-ray diffraction. See EXAMPLE 6. The triclinic cell parameters and calculated volume are: a=12.8951(14), b=16.9972(11), c=27.9468(13) Å, a=99.602(3), β=93.899(4), γ=104.059(3)°, V=5820.8(8) Å³. For Z=12 and a formula weight of 363.39 the calculated density is 1.244 g cm⁻³. The space group was determined to be P 1̅ (no. 2). A summary of the crystal data and crystallographic data collection parameters are provided in Table 2.

TABLE 2

| formula | C₂₀H₂₂NNaO₄ |
|---|---|
| formula weight | c363.39 |
| space group | P1 (No. 2) |
| a, Å | 12.8951(14) |
| b, Å | 16.9972(11) |
| c, Å | 27.9468(13) |
| a, deg | 99.602(3) |
| b, deg | 93.899(4) |
| g, deg | 104.059(3) |
| V, Å³ | 5820.8(8) |
| Z | 12 |
| d_{calc}, g cm⁻³ | 1.244 |
| crystal dimensions, mm | 0.44 × 0.25 × 0.15 |
| temperature, K | 150. |
| radiation (wavelength, Å) | Mo Kₐ (0.71073) |
| monochromator | graphite |
| linear abs coef, mm⁻¹ | 0.099 |
| absorption correction applied | empirical |
| transmission factors: min, max | 0.949, 0.985 |
| diffractometer | Nonius KappaCCD |
| h, k, l range | 0 to 15 −20 to 19 −33 to 32 |
| 2q range, deg | 4.17-50.04 |
| mosaicity, deg | 0.37 |
| programs used | SHELXTL |
| F₀₀₀ | 2304.0 |
| Weighting | 1/[s²(Fₒ²) + (0.0337P)² + 0.0000P] |
| | where P = (Fₒ² + 2F_{c}²)/3 |
| data collected | 39725 |
| unique data | 20104 |
| Rᵢₙₜ | 0.077 |
| data used in refinement | 17939 |
| cutoff used in R-factor | Fₒ² > 2.0s(Fₒ²) |
| calculations | |
| data with I > 2.0s(I) | 10814 |
| number of variables | 1453 |
| largest shift/esd in final cycle | 0.00 |
| R(Fₒ) | 0.047 |
| R_{w}(Fₒ²) | 0.090 |
| goodness of fit | 0.919 |

^{a}Otwinowski Z. & Minor, W. Methods Enzymol., 1997, 276, 307.

The quality of the structure obtained is high, as indicated by the R-value of 0.047 (4.7%). Usually R-values in the range of 0.02 to 0.06 are quoted for the most reliably determined structures.

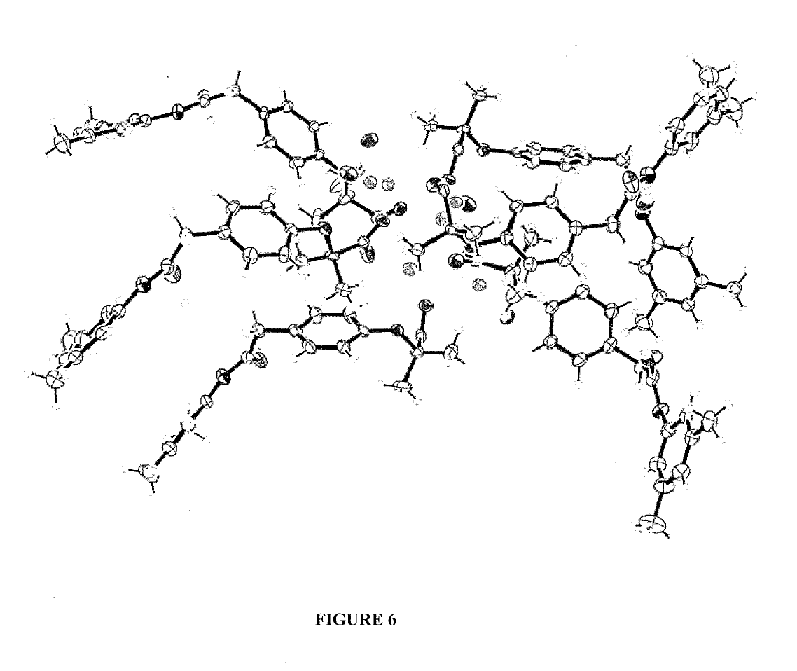

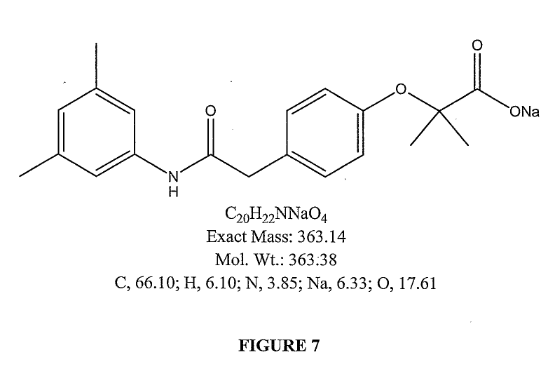

An ORTEP (See Johnson, C. K. ORTEPIII, Report ORNL-6895, Oak Ridge National Laboratory, TN, U.S.A. 1996. OPTEP-3 for Windows V1.05, Farrugia, L. J., J. Appl. Cryst. 1997, 30, 565.) drawing of Form A is shown in FIG. 6 (atoms are represented by 50% probability anisotropic thermal ellipsoids). The single crystal structure is the same as the proposed structure seen in FIG. 7. The asymmetric unit shown in FIG. 6 contains six efaproxiral molecules coordinating to six sodium cations. This is a very unusual number of molecules in the asymmetric unit and is the result of the six different coordination environments of the sodium atoms.

While the structure of Form A is quite complex, the structure can be best described as channels of sodium atoms linked together by the carboxylic acid anions of the efaproxiral molecules.

A calculated XRPD pattern of Form A was generated from the single crystal data and compared to the experimental pattern of Form A. All peaks in the experimental patterns are represented in the calculated XRPD pattern, indicating the bulk material is likely a single phase. The slight shifts in peak location are likely due to the fact that the experimental powder pattern was collected at ambient temperature, and the single crystal data was collected at 150 K. Low temperatures are used in single crystal analysis to improve the quality of the structure.

In summary, the single crystal structure of Form A was determined to confirm the molecular structure. The space group was determined to be P 1̅ (no. 2). The structure of Form A consists of six efaproxiral molecules and six sodium atoms forming sodium oxide channels running along the crystallographic 110 direction. All peaks in the experimental patterns are represented in the calculated XRPD pattern, indicating the bulk material is likely a single phase.

The space group of Form B was determined to be Triclinic P-1 based on XRPD measurements.

Hydrates of Efaproxiral Sodium

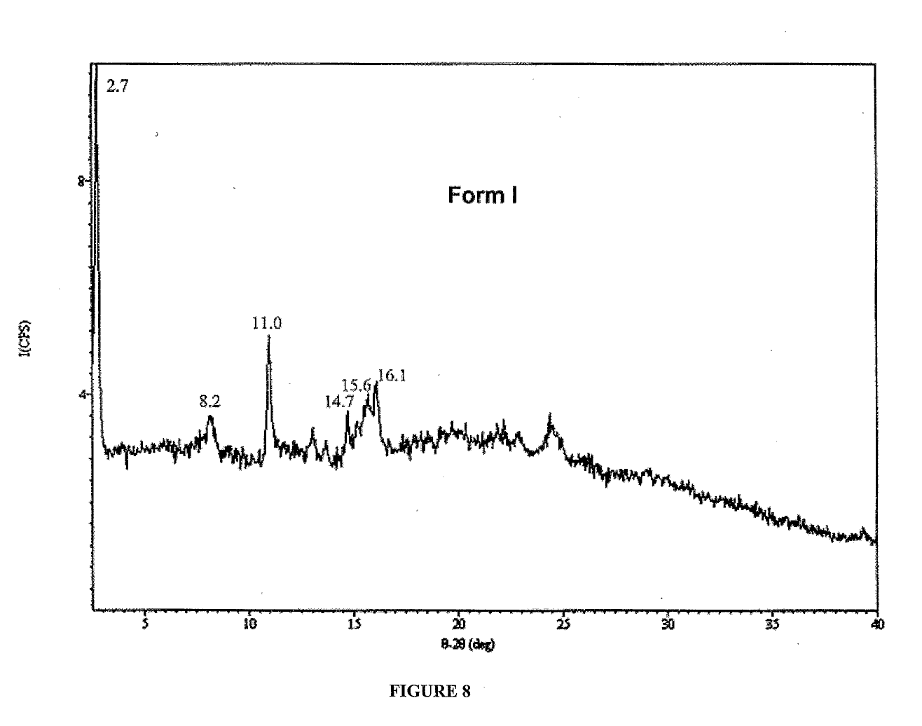

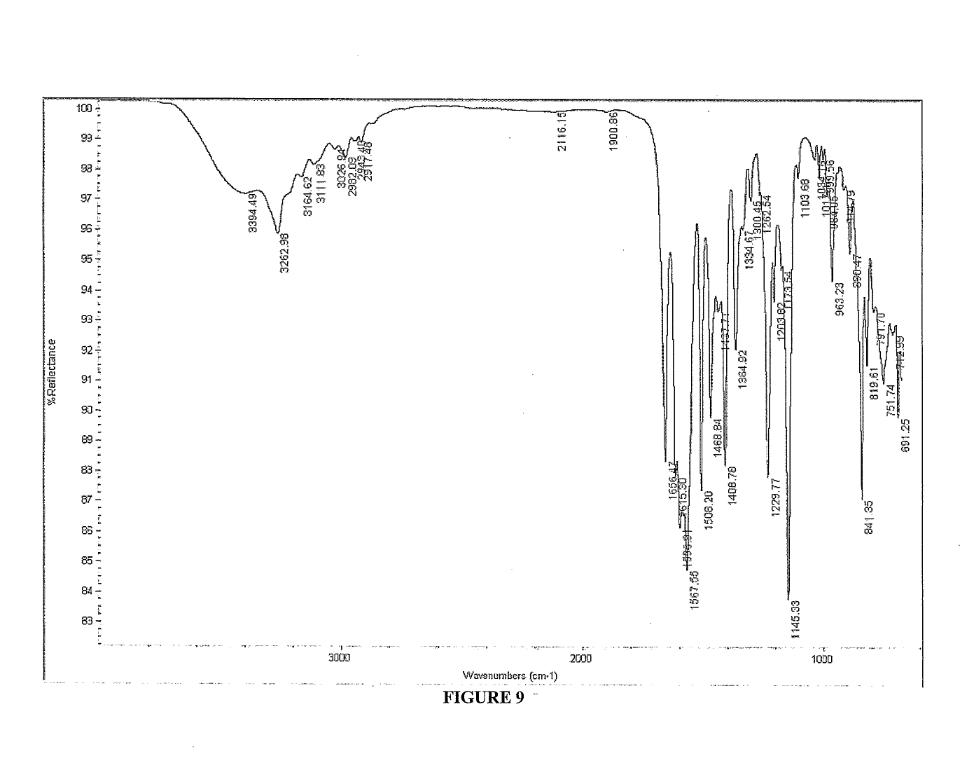

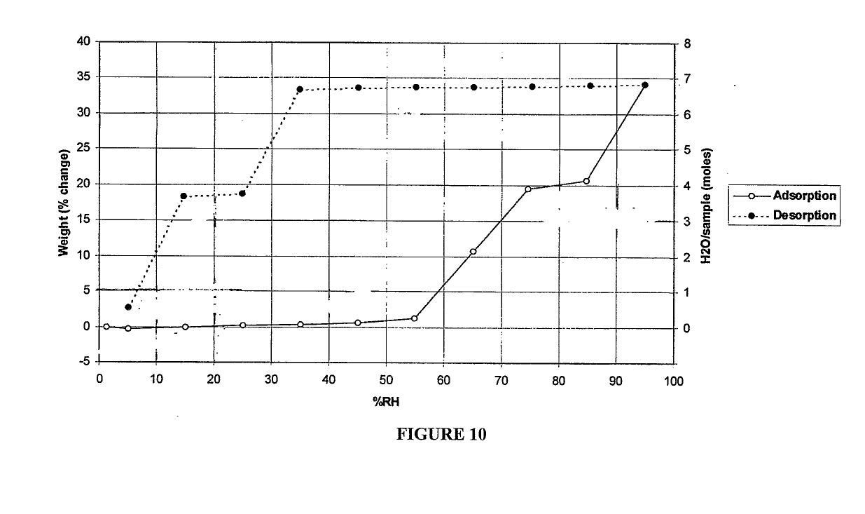

When Form A is incubated at high relative humidity, a new crystalline hydrate of efaproxiral sodium is formed, Form I (see EXAMPLE 7). The XRPD pattern of Form I is presented in FIG. 8, and the FTIR spectrum of Form I is presented in FIG. 9. Based on a weight gain of approximately 20% (which is equivalent to about 4 moles of water/mole efaproxiral sodium) under such conditions, Form I appears to be a tetrahydrate (see also EXAMPLE 8 and FIG. 10). Form I may also be obtained by limited dehydration of Form J (below) (see also EXAMPLE 8 and FIG. 10).

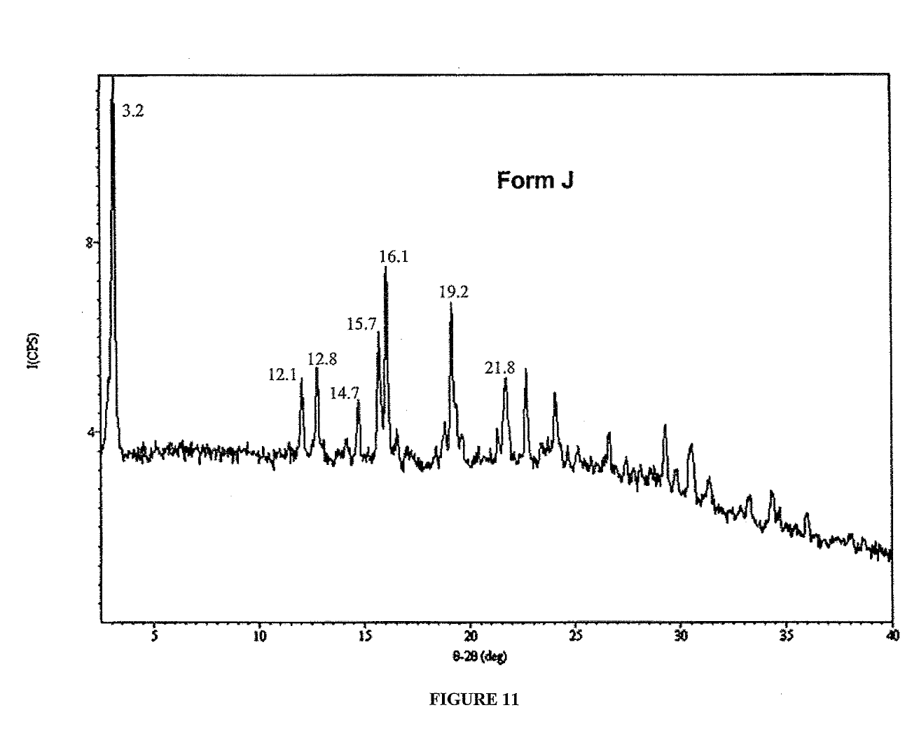

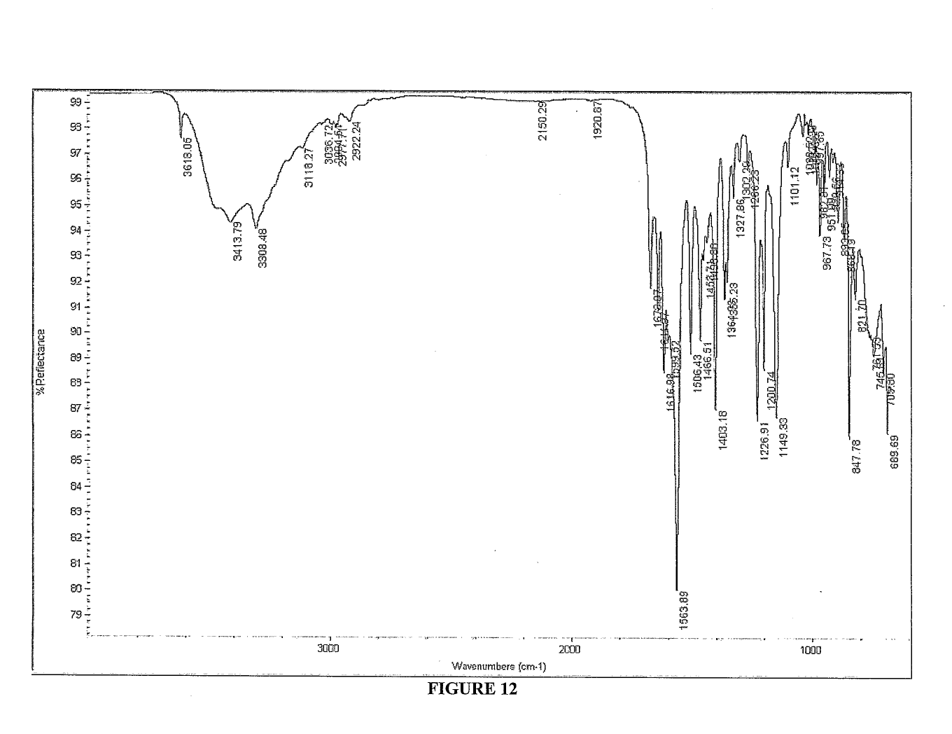

When Form A is slurried in water (see EXAMPLE 9), a new crystalline hydrate of efaproxiral sodium, Form J, is formed. The XRPD pattern of Form J is presented in FIG. 11, and the FTIR spectrum of Form J is presented in FIG. 12. When Form A is exposed to approximately 95% humidity the material experiences a weight gain of approximately 34% (equivalent to approximately 7 moles of water/mole efaproxiral sodium), suggesting that Form J is a heptahydrate (see EXAMPLE 8 and FIG. 10). Limited dehydration of Form J appears to yield Form I (see EXAMPLE 8 and FIG. 10).

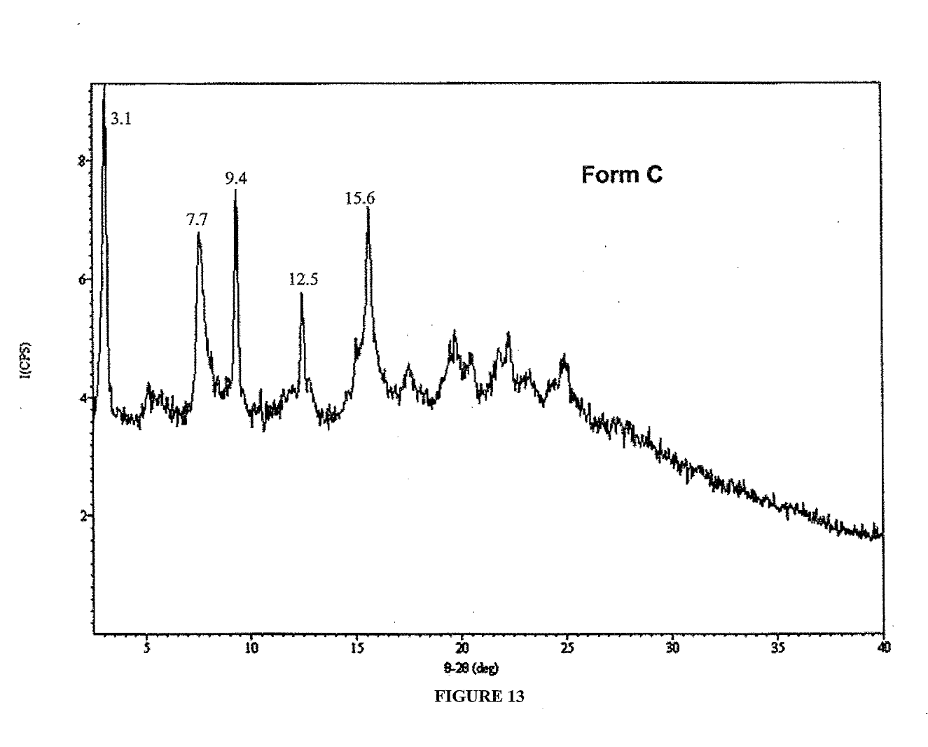

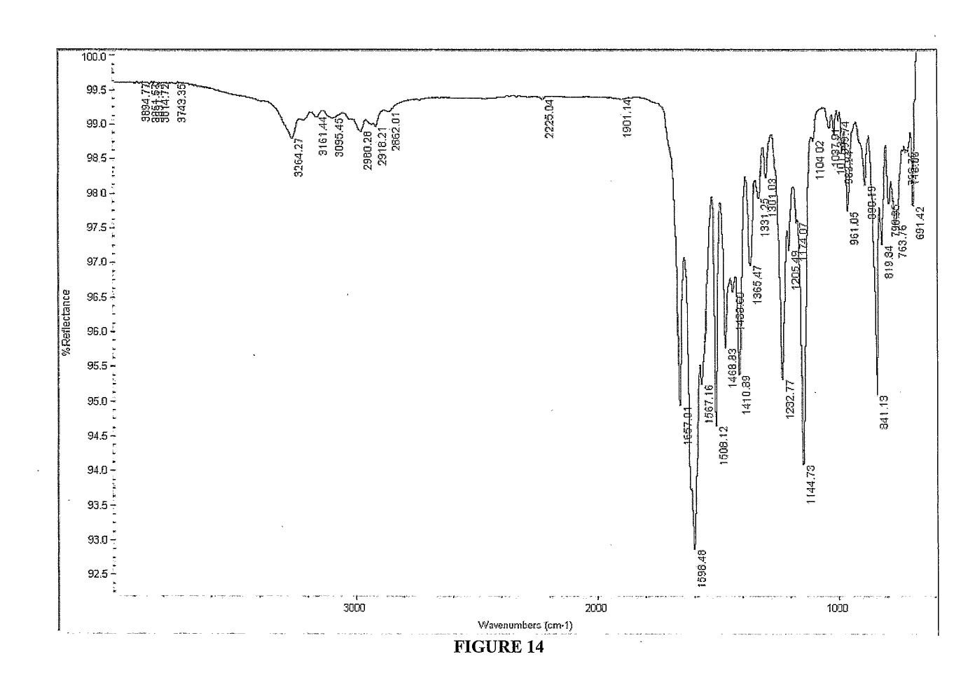

When either Form I or Form J is dehydrated (see EXAMPLE 10 and EXAMPLE 11), a new crystalline hydrate of efaproxiral sodium, Form C, is formed. The XRPD pattern of Form C is presented in FIG. 13, and the FTIR spectrum of Form C is presented in FIG. 14. Form C is a variable hydrate with less than four moles of water per mole of efaproxiral sodium.

Table 3 below tabulates certain angular XRPD peak positions in 20 for Form I (FIG. 8), Form J (FIG. 11) and Form C (FIG. 13) when obtained from a copper radiation source (CuKα λ=1.54 Å).

TABLE 3

| Form C | Form I | Form J |
|---|---|---|
| 2θ (degrees) | 2θ (degrees) | 2θ (degrees) |
| 3.09 | 2.74 | 3.15 |
| 7.67 | 8.2 | 12.06 |
| 9.36 | 10.96 | 12.77 |
| 12.47 | 14.72 | 14.73 |
| 15.63 | 15.67 | 15.73 |
| | 16.07 | 16.07 |
| | 24.55 | 19.16 |
| | | 21.72 |
| | | 22.7 |
| | | 24.09 |
| | | 29.28 |

Based on peak positions, and to some extent on peak intensities also, Form I possesses a characteristic XRPD peak at 11.0±0.2° in 2θ in contrast to the XRPD pattern of both Form J and Form C. Form J possesses a characteristic XRPD peak at 19.2±0.2° in 2θ in contrast to the XRPD pattern of both Form C and Form I. Form J also possesses a pair of characteristic peaks at 12.1±0.2° and 12.8±0.2° in 2θ in contrast to the XRPD patterns of Form I and Form C. Form C possesses a characteristic XRPD peak at 7.7±0.2° in 2θ in contrast to the XRPD patterns of Form I and Form J. Hence, it is possible to differentiate between the three hydrated forms of efaproxiral sodium using at least the aforementioned characteristic peaks, or a combination of the aforementioned characteristic peaks and the other peaks in Table 2.

Alternatively, Form J may also be identified by a unique FTIR absorption band at 3618±2 cm⁻¹; or by a unique FTIR absorption band at 1921±2 cm⁻¹, or by a unique FTIR absorption band at 1028±2 cm⁻¹; or by any combination of these unique FTIR absorption bands. Form C may also be identified by a unique FTIR absorption band at 2225±2 cm⁻¹.

Ethanol Solvates

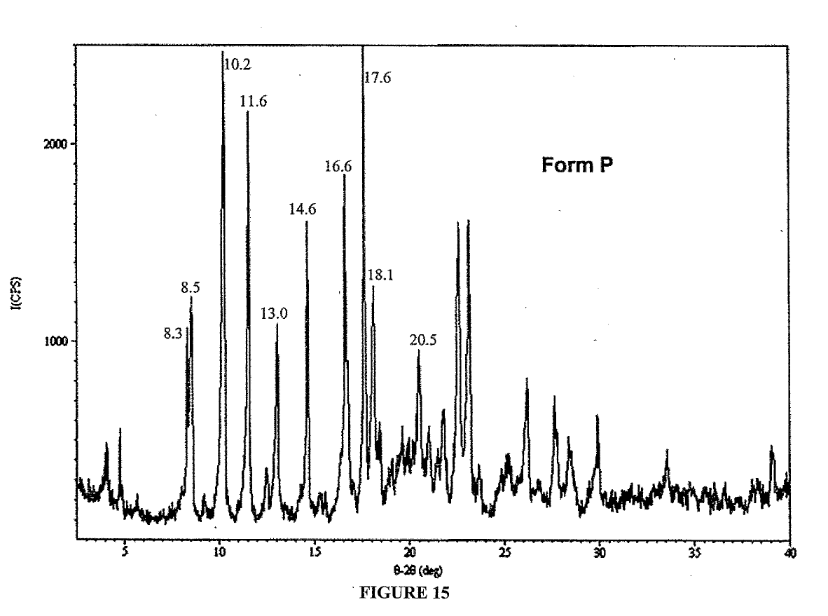

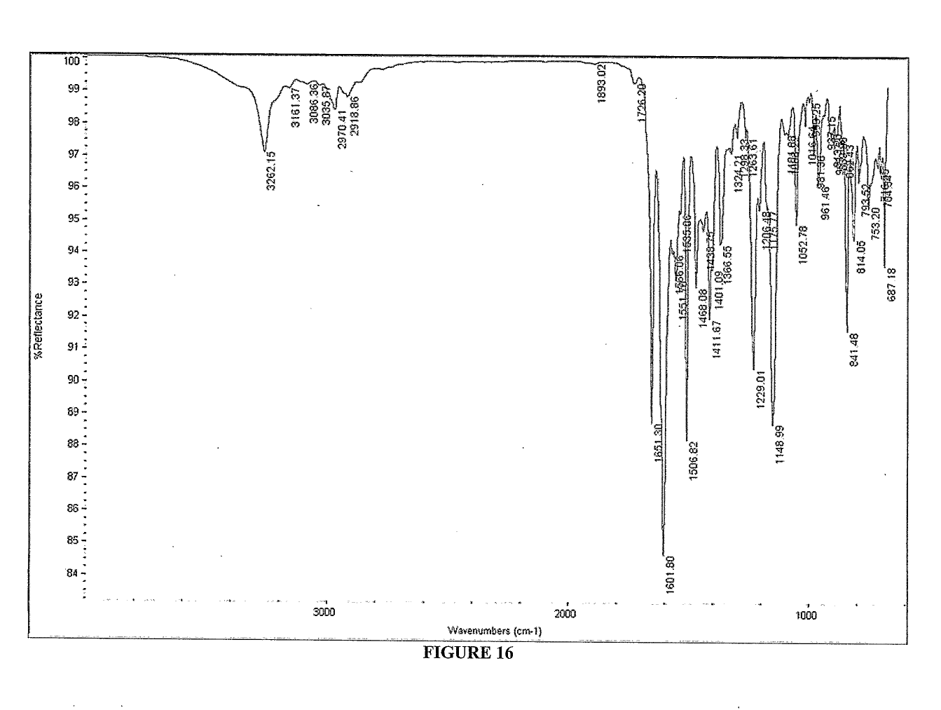

Form P is a new crystalline form of efaproxiral sodium that may be formed by dissolving efaproxiral sodium in ethanol and cooling the solution to precipitate crystalline efaproxiral sodium (see the final recrystallization step in EXAMPLE 1). The XRPD pattern of Form P is presented in FIG. 15, and the FTIR spectrum of Form P is presented in FIG. 16.

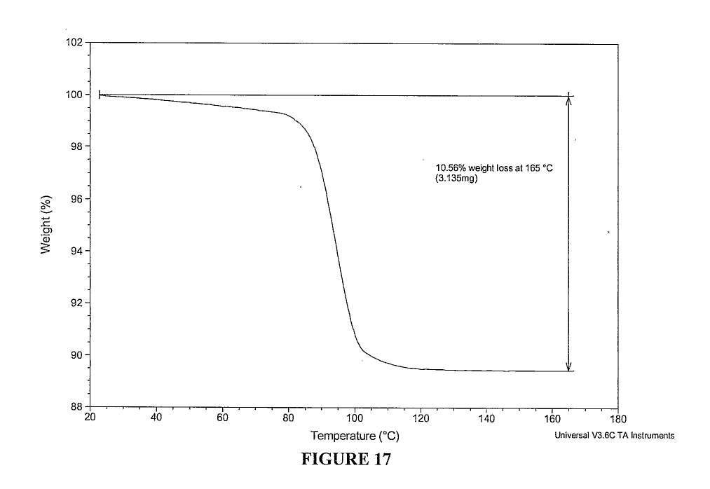

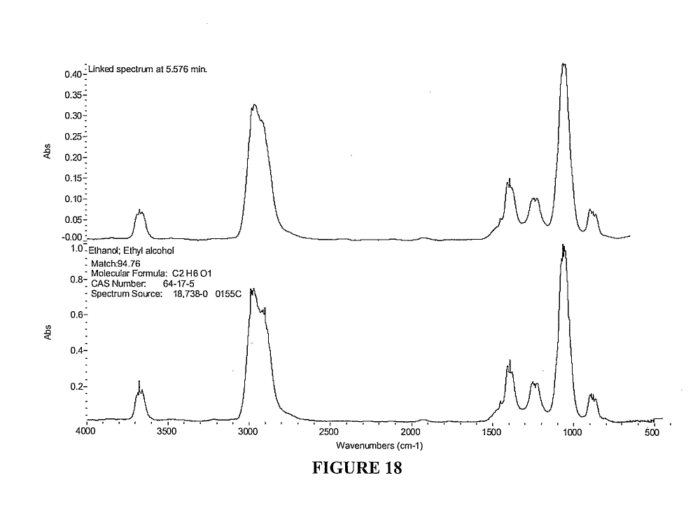

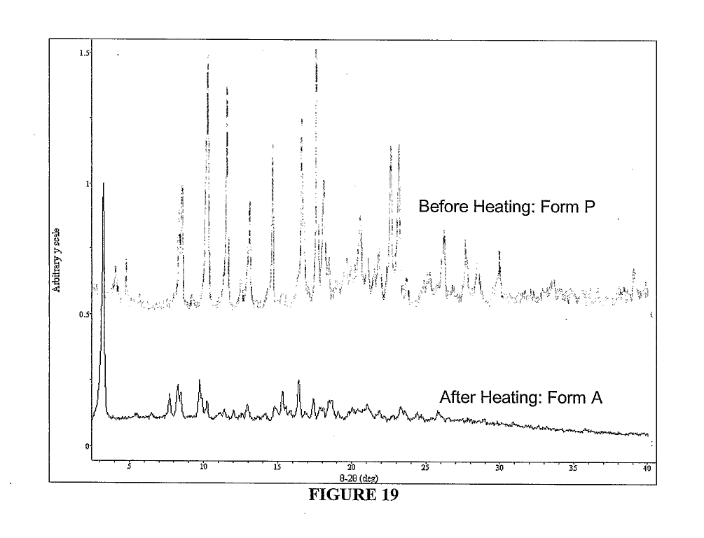

TGA of Form P indicates that this form undergoes an approximately 10.6% weight loss as the temperature is raised from ambient to 165° C. (FIG. 17). This is equivalent to the loss of about 1 mole of ethanol per mole of efaproxiral sodium, indicating that Form P is a monoethanolate. The volatile that is evaporated from Form P during heating was analyzed by FTIR spectroscopy and was found to be ethanol, again suggesting that Form P is an ethanolate in which the ethanol in the crystal lattice may be removed by heating (FIG. 18). The solid material remaining after Form P was heated to 156° C. was then analyzed by XRPD and was found to be Form A (FIG. 19). Therefore, it is believed that when ethanol is removed from the crystal lattice of Form P by heating, the crystal adopts the Form A structure.

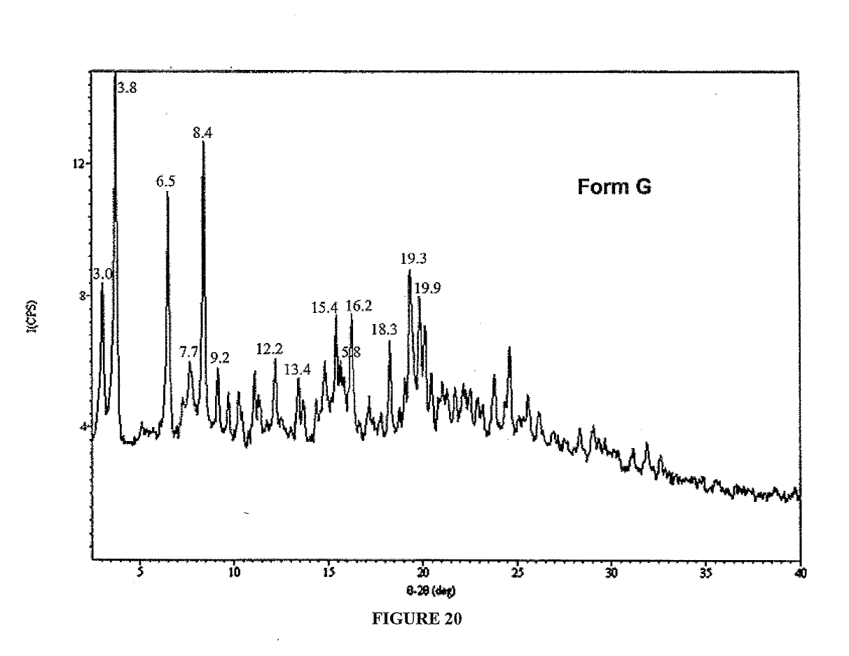

Form G is a new crystalline form of efaproxiral sodium that forms transiently during acetone/ethanol recrystallization (see EXAMPLE 12) and is also isolated when Form A is stirred in slurry in acetonitrile and ethanol (see EXAMPLE 13). Since Form G was isolated from two different solvent systems that both contained ethanol, it is likely that Form G is an ethanolate of efaproxiral sodium. The XRPD pattern of Form G is presented in FIG. 20.

Table 4 below tabulates certain angular XRPD peak positions in 20 for Form P (FIG. 15) and Form G (FIG. 20) when obtained from a copper radiation source (CuKα λ=1.54 Å).

TABLE 4

| Form P | Form G |
|---|---|
| 2θ | 2θ |
| 8.32 | 3.04 |
| 8.52 | 3.76 |
| 10.23 | 6.51 |
| 11.55 | 7.72 |
| 13.04 | 8.43 |
| 14.63 | 9.16 |
| 16.6 | 12.2 |
| 17.61 | 13.38 |
| 18.07 | 15.41 |
| 20.51 | 15.83 |
| 22.58 | 16.2 |
| 23.12 | 18.26 |
| 26.16 | 19.33 |
| 27.62 | 19.85 |
| | 20.09 |
| | 21.07 |
| | 22.09 |
| | 23.79 |
| | 24.59 |

Based on peak positions, and to some extent on peak intensities also, Form G has a pair of characteristic XRPD peaks at 3.0±0.2° and 3.8±0.2° in 2θ in contrast to polymorphs A, B, C, F, I, J, and Q. Form G possesses characteristic XRPD peaks at 3.01±0.2°, 3.8±0.2°, 6.5±0.2°, 9.2±0.2°, and 12.2±0.2° in 2θ in contrast to Form P. Form P possesses characteristic XRPD peaks at 10.2±0.2°, 16.7±0.2°, and 17.6±0.2° in 2θ in contrast to Form G. Hence it is possible to differentiate between the ethanolate forms of efaproxiral sodium, and also between Form G and all other polymorphs, using at least the aforementioned characteristic peaks, or a combination of the aforementioned characteristic peaks and the other peaks in Table 4.

Alternatively, Form P may also be identified by a unique FTIR absorption band at 3086±12 cm⁻¹; or by a unique FTIR absorption band at 1088±2 cm⁻¹, or by a unique FTIR absorption band at 903±2 cm⁻¹; or by any combination of these unique FTIR absorption bands.

Acetone Solvate

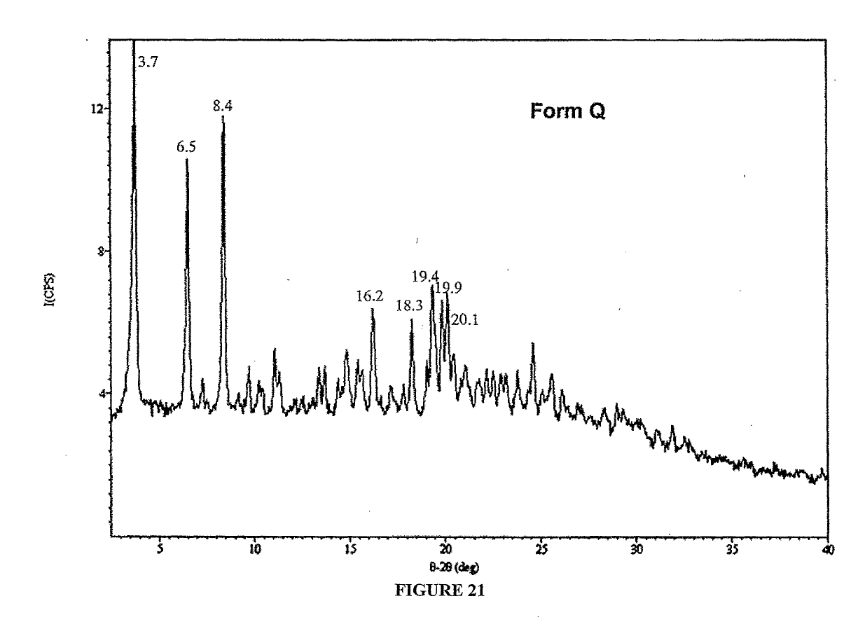

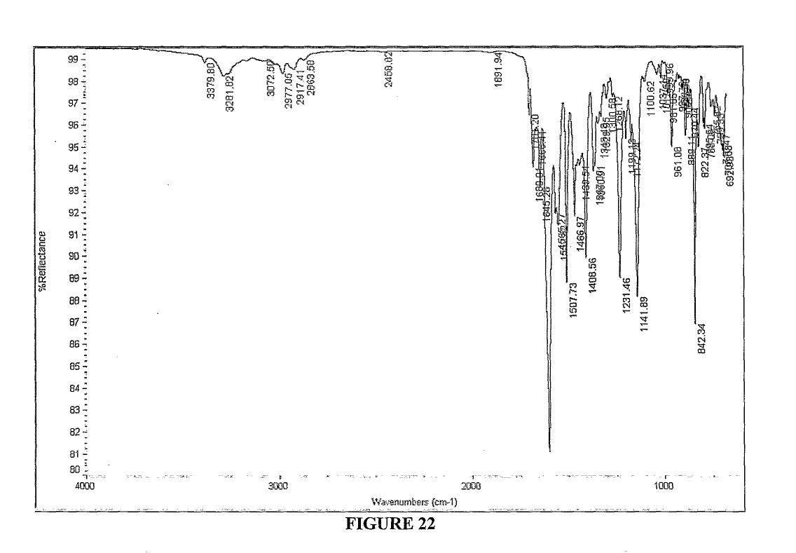

Form Q is a new crystalline form of efaproxiral sodium that may be formed by dissolving efaproxiral sodium in ethanol with stirring at elevated temperature, adding acetone to the solution with continued stirring, then cooling the solution below 25° C. with continued stirring. (see EXAMPLE 12). The XRPD pattern of Form Q is presented in FIG. 21, and the FTIR spectrum of Form Q is presented in FIG. 22. Table 5 below tabulates certain angular XRPD peak positions in 2θ for Form Q (FIG. 21) when obtained from a copper radiation source (CuKα λ=1.54 Å).

TABLE 5
Form Q
2θ (degrees)

3.74
6.5
8.42
16.23
18.27
19.38
19.85
20.12
24.6

Alternatively, Form Q may also be identified by a unique FTIR absorption band at 3380±2 cm⁻¹; or by a unique FTIR absorption band at 1701±2 cm⁻¹, or by a unique FTIR absorption band at 1645±2 cm⁻¹; or by any combination of two or more of these unique FTIR absorption bands.

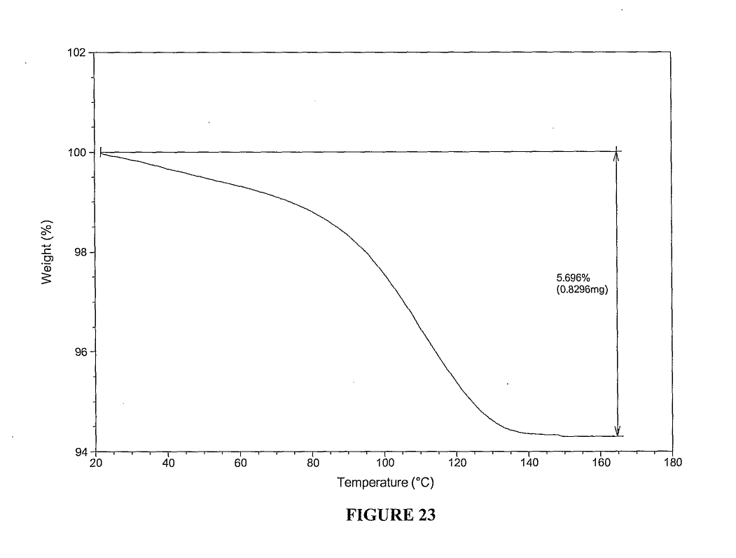

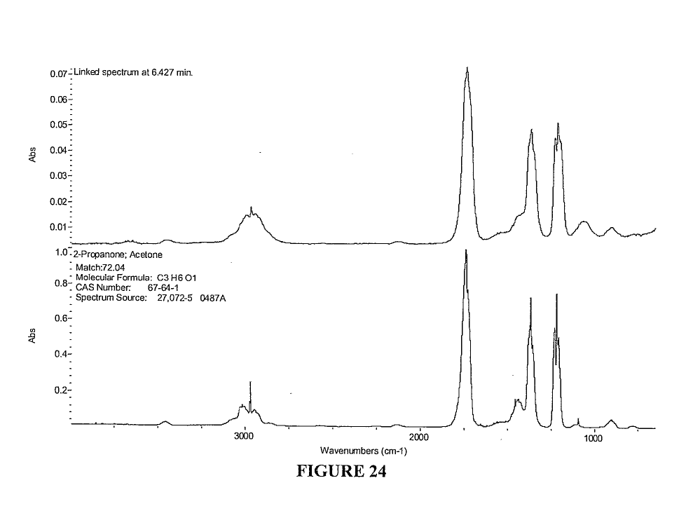

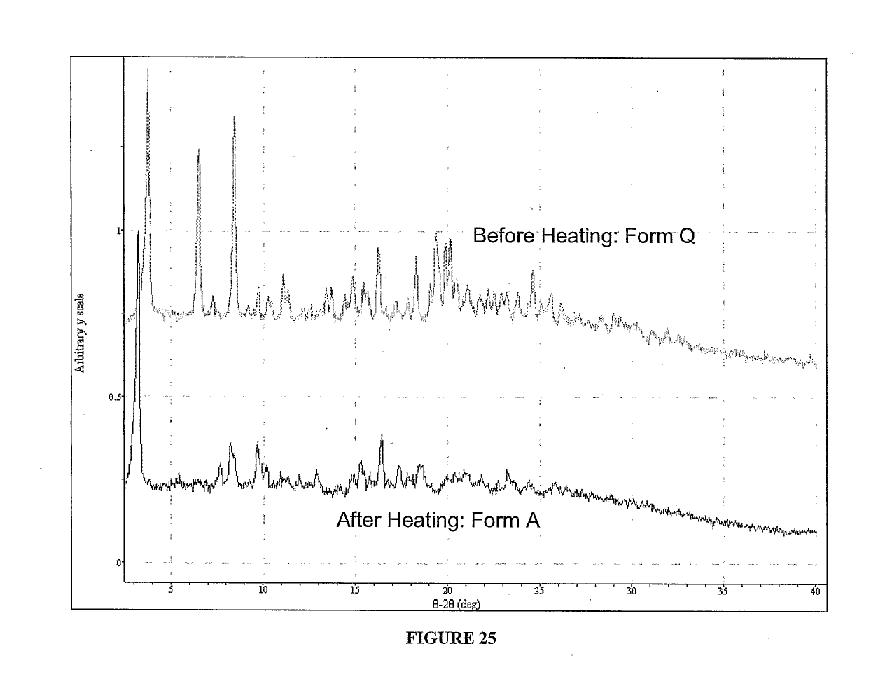

TGA of Form Q indicates that this form undergoes an approximately 5.7% weight loss as the temperature is raised from ambient to about 165° C. (FIG. 23). This is equivalent to the loss of about ½ to about ⅓ mole of acetone per mole of efaproxiral sodium, indicating that Form Q is likely to be a solvate of acetone. It is likely that Form Q comprises between 1 mole and ½ mole of acetone per mole of efaproxiral sodium (it is believed that acetone is lost from Form Q during preparation for TGA, and that therefore the measured loss of ½ to about ⅓ mole of acetone per mole of efaproxiral sodium observed during TGA does not fully reflect the amount of acetone in Form Q). The volatile that is removed from Form Q during heating was analyzed by FTIR spectroscopy and was found to be acetone, again suggesting that Form Q is an acetone solvate in which the acetone in the crystal lattice may be removed by heating (FIG. 24). The solid material remaining after Form Q was heated to about 165° C. was then analyzed by XRPD and was found to be Form A (FIG. 25). Therefore, it is believed that when acetone is removed from the crystal lattice of Form Q by heating, the crystal adopts the Form A structure. Thus, in EXAMPLE 4 it is believed that, prior to drying, the recovered crystals are Form Q, and that after extensive drying the crystals are Form A. Moreover, when Form Q is dried at 70° C. under vacuum, it forms Form B rather than Form A. Thus, during the final recrystallization and drying step of EXAMPLE 4, three polymorphs are likely to arise: Form Q is formed initially during the recrystallization, which then converts to Form B during initial drying, and finally Form A upon extensive drying. Depending upon the extent of drying performed in order to isolate solid efaproxiral sodium, the final solid form may thus be Form A or Form B, both of which are unsolvated.

Methanol Solvate

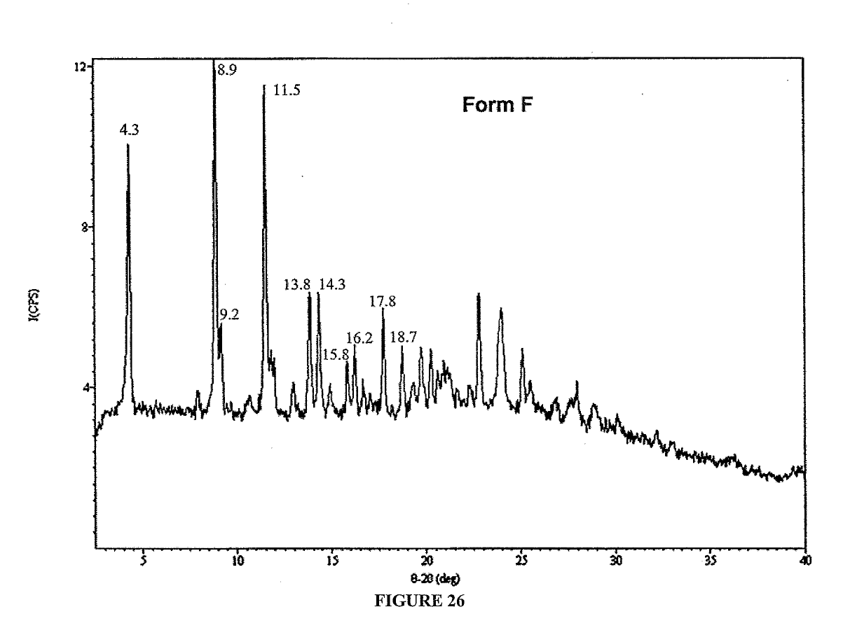

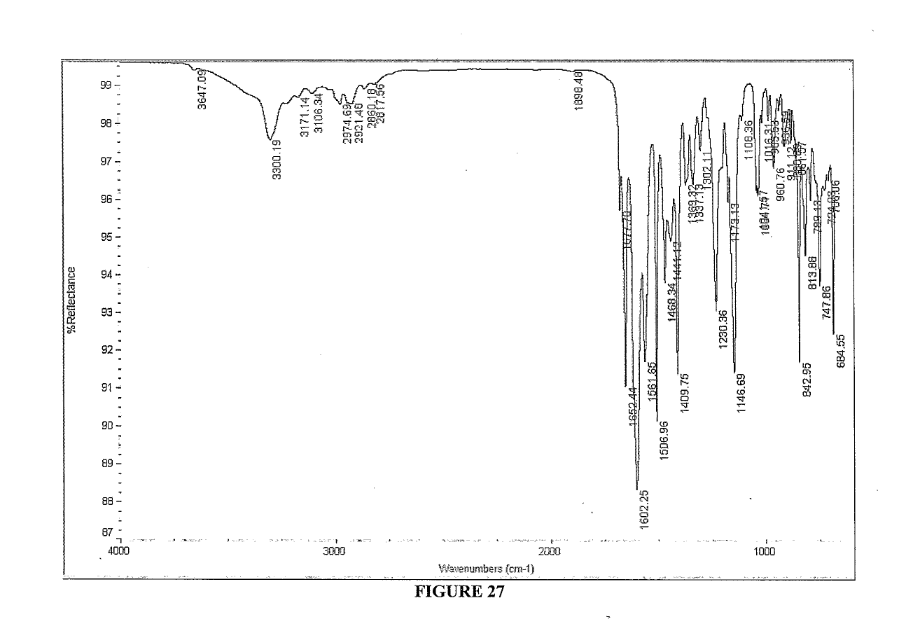

Form F is a new crystalline form of efaproxiral sodium that forms when efaproxiral sodium is dissolved in methanol and then the methanol is removed, for example by vapor diffusion (see EXAMPLE 14). Form F comprises about 1 mole of methanol per mole of efaproxiral sodium. The XRPD pattern of Form F is presented in FIG. 26 and the FTIR spectrum of Form F is presented in FIG. 27. Table 6 below tabulates certain angular XRPD peak positions in 20 for Form F (FIG. 26) when obtained from a copper radiation source (CuKα λ=1.54 Å).

TABLE 6
Form F
2θ (degrees)

4.31
8.91
9.19
11.53
13.83
14.34
15.84
16.23
17.75
18.74
22.8
23.96
25.1

Alternatively, Form F may also be identified by a unique FTIR absorption band at 747±2 cm⁻¹, or by a unique FTIR absorption band at 1053±2 cm⁻¹, or by a unique FTIR absorption band at 1338±2 cm⁻¹, or by a unique FTIR absorption band at 1562±2 cm¹ or by any combination of these unique FTIR absorption bands.

The crystal structure of Form F efaproxiral sodium was determined using single crystal X-ray diffraction. See EXAMPLE 15. The triclinic cell parameters and calculated volume are: a=11.408(4) Å, b=22.455(8) Å, c=27.318(7) Å, a=112.087(16)°, β=101.038(14)°, γ=92.02(2)°, V=6320(3) Å³. For Z=12 and formula weight of 395.43, the calculated density is 1.247 g/cm³. The space group was determined to be P 1̅ (No. 2). A summary of the crystal data and crystallographic data collection parameters is provided in Table 7.

TABLE 7

| formula | C₂₁H₂₆NNaO₅ |
|---|---|
| formula weight | 395.43 |
| space group | P i̅ (No. 2) |
| a, Å | 11.408(4) |
| b, Å | 22.455(8) |
| c, Å | 27.318(7) |
| a, deg | 112.087(16) |
| b, deg | 101.038(14) |
| g, deg | 92.02(2) |
| V, Å³ | 6319(3) |
| Z | 12 |
| d_{calc}, g cm⁻³ | 1.247 |
| crystal dimensions, mm | 0.38 × 0.35 × 0.11 |
| temperature, K | 150. |
| radiation (wavelength, Å) | Mo Kₐ (0.71073) |
| monochromator | graphite |
| linear abs coef, mm⁻¹ | 0.099 |
| absorption correction applied | empirical^{a} |
| transmission factors: min, max | unknown, 0.99 |
| diffractometer | Nonius KappaCCD |
| h, k, l range | 0 to 12 −25 to 25 −30 to 29 |
| 2θ range, deg | 4.30-47.64 |
| mosaicity, deg | 2.95 |
| programs used | SHELXTL |
| F₀₀₀ | 2520.0 |
| Weighting | 1/[σ²(Fₒ²) + (0.0651P)² + 2.9980P] |
| | where P = (Fₒ² + 2F_{c}²)/3 |
| data collected | 50155 |
| unique data | 18687 |
| Rᵢₙₜ | 0.136 |
| data used in refinement | 13421 |
| cutoff used in R-factor | Fₒ² > 2.0σ(Fₒ²) |
| calculations | |
| data with I > 2.0σ(I) | 7593 |
| refined extinction coef | 0.0014 |
| number of variables | 1592 |
| largest shift/esd in final cycle | 0.00 |
| R(Fₒ) | 0.073 |
| R_{w}(Fₒ²) | 0.151 |
| goodness of fit | 1.030 |

^{a}Otwinowski Z. & Minor, W. Methods Enzymol., 1997, 276, 307.

Usually R-values in the range of 0.02 to 0.06 are quoted for the most reliably determined structures (See Glusker, Jenny Pickworth; Trueblood, Kenneth N. Crystal Structure Analysis: A Primer, 2^{nd} ed.; Oxford University press: New York, 1985; p. 87.) While the R-value of 0.073 (7.3%) is slightly outside of the reported range, the quality of the structure obtained is high.

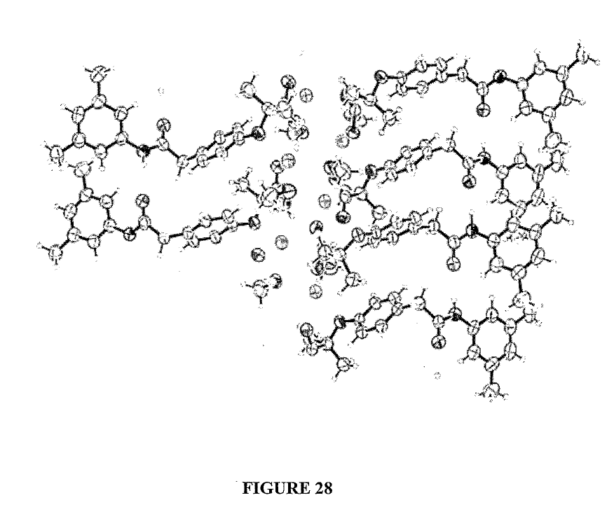

An ORTEP (See Johnson, C. K. ORTEPIII, Report ORNL-6895, Oak Ridge National Laboratory, TN, U.S.A. 1996. OPTEP-3 for Windows V1.05, Farrugia, L. J., J. Appl. Cryst. 1997, 30, 565.) drawing of Form F efaproxiral sodium is shown in FIG. 28 (atoms are represented by 50% probability anisotropic thermal ellipsoids). The single crystal structure is the same as the proposed structure seen in FIG. 7. The asymmetric unit shown in FIG. 28 contains six efaproxiral and methanol molecules coordinated to six sodium cations. This is a very unusual number of molecules in the asymmetric unit.

The structure of Form F displays a complex coordination scheme to the sodium ions. Again the structure can be best described as channels of sodium atoms linked together by the carboxylic acid anions of the efaproxiral molecules and methanol molecules. The methanol solvent molecules are coordinating to the sodium ions in two different configurations. One type of methanol is coordinated to a single sodium ions while the other is bridging two sodium ions. Each sodium ion is capped by one methanol molecule and is bridging to two other molecules. The solvent is closely associated to the sodium oxide channels but can be removed by heating the sample to 80° C. for approximately 3 hours.

A calculated XRPD pattern of Form F was generated from the single crystal data and compared to the experimental pattern of Form F. All peaks in the experimental patterns are represented in the calculated XRPD pattern, indicating the bulk material is likely a single phase. The slight shifts in peak location are likely due to the fact that the experimental powder pattern was collected at ambient temperature, and the single crystal data was collected at 150 K. Low temperatures are used in single crystal analysis to improve the quality of the structure.

In summary, the single crystal structure of Form F efaproxiral sodium was determined to confirm the molecular structure. The space group was determined to be P 1̅ (no. 2). The structure of Form F efaproxiral sodium consists of six efaproxiral and methanol molecules coordinating to six sodium atoms resulting in a sodium oxide channel that runs along the crystallographic 101 direction. All peaks in the experimental patterns are represented in the calculated XRPD pattern, indicating the bulk material is likely a single phase.

Amorphous Efaproxiral Sodium

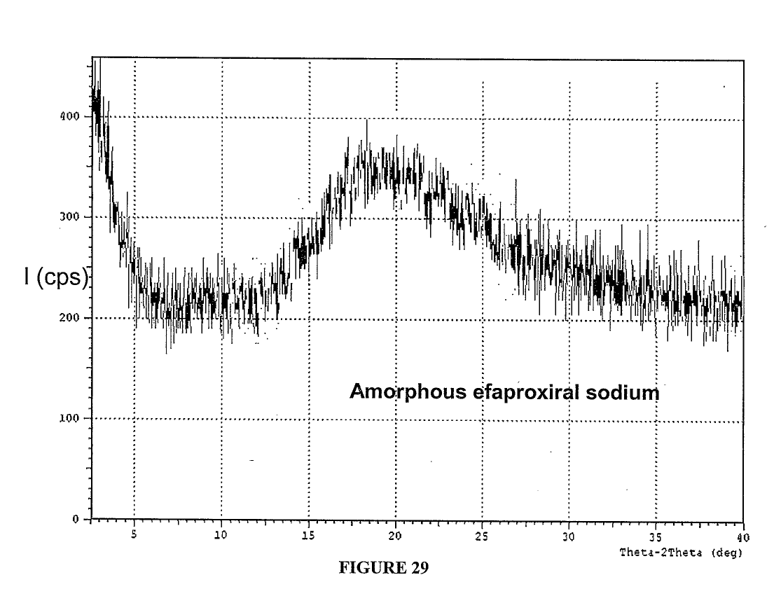

Amorphous efaproxiral sodium was obtained by freeze-drying efaproxiral sodium dissolved in dioxane/water (see EXAMPLE 16). Amorphous material was also obtained when the relative humidity (RH) surrounding a sample of Form J efaproxiral sodium was decreased from nearly 100% to 5% (see EXAMPLE 8). The XRPD pattern of amorphous efaproxiral sodium is presented in FIG. 29 and, as is typical for amorphous material, lacks the sharp reflectance peaks observed in crystalline material.

Pharmaceutical Formulations

For the most effective administration of drug substance of the present invention, it is preferred to prepare a pharmaceutical formulation (also known as the “drug product”) preferably in unit dose form, comprising one or more of the efaproxiral sodium polymorphs of the present invention and/or the amorphous efaproxiral sodium of the invention, and one or more pharmaceutically acceptable carrier, diluent, or excipient. With reference to efaproxiral sodium, suitable formulations are described in copending U.S. Patent Application Publication No. 20030232887 A1, incorporated by reference herein in its entirety.

A pharmaceutical formulation may, without being limited by the teachings set forth herein, include a solid form of the present invention which is blended with at least one pharmaceutically acceptable excipient, diluted by an excipient or enclosed within such a carrier that can be in the form of a capsule, sachet, tablet, buccal, lozenge, paper, or other container. When the excipient serves as a diluent, it may be a solid, semi-solid, or liquid material which acts as a vehicle, carrier, or medium for the efaproxiral sodium polymorph(s). Thus, the formulations can be in the form of tablets, pills, powders, elixirs, suspensions, emulsions, solutions, syrups, capsules (such as, for example, soft and hard gelatin capsules), suppositories, sterile injectable solutions, and sterile packaged powders.

Examples of suitable excipients include, but are not limited to, starches, gum arabic, calcium silicate, microcrystalline cellulose, polyvinylpyrrolidone, cellulose, water, syrup, and methyl cellulose. The formulations can additionally include lubricating agents such as, for example, talc, magnesium stearate and mineral oil; wetting agents; emulsifying and suspending agents; preserving agents such as methyl- and propyl-hydroxybenzoates; sweetening agents; or flavoring agents. Polyols, buffers, and inert fillers may also be used. Examples of polyols include, but are not limited to: mannitol, sorbitol, xylitol, sucrose, maltose, glucose, lactose, dextrose, and the like. Suitable buffers encompass, but are not limited to, phosphate, citrate, tartrate, succinate, and the like. Other inert fillers which may be used encompass those which are known in the art and are useful in the manufacture of various dosage forms. If desired, the solid pharmaceutical compositions may include other components such as bulling agents and/or granulating agents, and the like. The compositions of the invention can be formulated so as to provide quick, sustained, controlled, or delayed release of the drug substance after administration to the patient by employing procedures well known in the art.

In the event that the above formulations are to be used for parenteral administration, such a formulation typically comprises sterile, aqueous and non-aqueous injection solutions comprising one or more efaproxiral sodium forms for which preparations are preferably isotonic with the blood of the intended recipient. These preparations may contain anti-oxidants, buffers, bacteriostats, and solute; which render the formulation isotonic with the blood of the intended recipient. Aqueous and non-aqueous suspensions may include suspending agents and thickening agents. The formulations may be present in unit-dose or multi-dose containers, for example, sealed ampules and vials. Extemporaneous injection solutions and suspensions may be prepared from sterile powders, granules, and tablets of the kind previously described.

As such, the pharmaceutical formulations of the present invention are preferably prepared in a unit dosage form, each dosage unit containing from about 5 mg to about 200 mg, more usually about 100 mg of the efaproxiral sodium form(s). In liquid form, dosage unit contains from about 5 to about 200 mg, more usually about 100 mg of the efaproxiral sodium form(s). The term “unit dosage form” refers to physically discrete units suitable as unitary dosages for human subjects/patients or other mammals, each unit containing a predetermined quantity of the efaproxiral sodium polymorph calculated to produce the desired therapeutic effect, in association with preferably, at least one pharmaceutically acceptable carrier, diluent, or excipient. EXAMPLE 17 below provide examples of aqueous formulations of efaproxiral sodium.

The following examples are provided for illustrative purposes only, and are not to be construed as limiting the scope of the claims in any way.

### EXAMPLES

### Example 1

### Preparation of Efaproxiral Sodium

Synthesis of Amidophenol (3)

1,4-hydroxyphenylacetic acid (200 kg) (2) was added to xylene (760 L) with stirring in either Hastelloy 276®, SS (316) or glass-lined SS reactors. To this mixture, 3,5-xylidine (3,5-dimethyl aniline) (178 L) (1) was added. The reaction mixture was heated to reflux and water was removed azeotropically as the reaction proceeded. Upon completion, the reaction mixture was distilled to provide amidophenol (3), which solidified upon cooling. To recrystallize, ethanol (1180 L) and methyl isobutyl ketone (MIBK) (56 L) were added to the solid and the mixture was refluxed until dissolution. Upon dissolution water was added (70° C., 490 L) and mixture was stirred and cooled slowly over 6 hours to about 0° C. The mixture was then stirred for at least one hour at this temperature. The mixture was then filtered, and the solid washed with 1:2 ethanol/water at 5° C., followed by a wash with xylene (452 L at 5° C.).

Synthesis of Efaproxiral Ethyl Ester (4)

Methyl isobutyl ketone (MIBK) (827 L) was added to the crystallized amidophenol (3) and the mixture was refluxed to azeotropically remove water. The reaction mixture was then cooled to below 70° C., and absolute ethanol (731 L) was added, followed by anhydrous potassium carbonate (668 kg) and ethyl 2-bromoisobutyrate (366 L). The reaction mixture was refluxed for at least 7 hours, then cooled to below 0° C. The mixture was filtered, and the solids were washed with MIBK such that the total volume of the wash plus the filtrate was 1208 L. The mixture was the distilled to remove the ethanol and the volume was adjusted with MIBK to about 2163 L. The MIBK mixture was extracted with dilute aqueous base (32 kg sodium bicarbonate in 604 L of water), followed aqueous acid (63 L in 572 liters of water, and water (3×700 L each). The mixture was then distilled to remove MIBK and cooled to about 35° C. Heptane (about 572 L) was added and the solution was stirred while additional heptane (approximately 1145 L) was slowly added over the course of one hour. The mixture was then cooled to about 12° C., stirred for at least 2 hours and then filtered. The solid, efaproxiral ethyl ester (4) was washed with heptane (318 L).

Synthesis of Efaproxiral Sodium (5)

Absolute ethanol (880 L) was first mixed with water (19 L), followed by the addition of sodium hydroxide (36 kg). This mixture was filtered, efaproxiral ethyl ester (4) was added and the reaction mixture was refluxed for at least 3 hours. Sodium hydroxide (10 N, 1 molar equivalent) was then added and reflux was maintained for at least 5 hours after the last addition. The mixture was then concentrated by distillation, and absolute ethanol (1056 L) was added. The water content was less than 0.5%. The reaction mixture was then cooled to about 40° C., then 35° C., and stirred for at least 2 hours. The mixture was then concentrated under vacuum to about 1408 L, cooled to about 10° C., and stirred for at least 5 hours. The mixture was then filtered and the solid, which consisted primarily of efaproxiral sodium (5), was washed with ethanol (282 L at 10° C.).

Purification of Efaproxiral Sodium (5) by Extraction with Methyl Isobutyl Ketone (MIBK)

Purified water (1658 L) was added to the efaproxiral sodium (5) (325 kg). The mixture was distilled under vacuum at a maximum temperature of 50° C. until about 423 L of solvent was removed. Another 423 L of purified water was then added and the aqueous solution was extracted with MIBK (390 L, below 30° C.). The organic phase was discarded, the aqueous phase was extracted again with MIBK (228 L, below 30° C.) and the organic phase was discarded.

### Example 2

### FTIR Protocol

The FTIR spectra were acquired on a Magna-IR 860° Fourier transform infrared (FT-IR) spectrophotometer (Thermo Nicolet) equipped with an Ever-Glo mid/far IR source, an extended range potassium bromide (KBr) beamsplitter, and a deuterated triglycine sulfate (DTGS) detector. An attenuated total reflectance (ATR) accessory (the Thunderdome™, Thermo Spectra-Tech), with a germanium (Ge) crystal was used for data acquisition. The spectrum represents 256 co-added scans collected at a spectral resolution of 4 cm⁻¹. A background data set was acquired with a clean Ge crystal. Wavelength calibration was performed using polystyrene.

### Example 3

### XRPD Protocol

XRPD analyses were carried out on a Shimadzu XRD-6000 X-ray powder diffractometer using Cu Kα radiation. The instrument is equipped with a long fine focus X-ray tube. The tube voltage and amperage were set at 40 kB and 40 mA, respectively. The divergence and scattering slits were set at 1° and the receiving slit was set at 0.15 mm. Diffracted radiation was detected by a NaT scintillation detector. A theta-two theta continuous scan at 3′/min (0.4 sec/0.02° step) from 2.5 to 40 °2θ was used. A silicon standard was analyzed each day to check the instrument alignment. Samples were analyzed with an aluminum/silicon sample holder.

### Example 4

### Purification of Efaproxiral Sodium by Recrystallization with Acetone/Ethanol

Efaproxiral sodium in aqueous solution produced according to EXAMPLE 1 above was concentrated under vacuum at a maximum temperature of 50° C. to the maximum extraction of solvent, after which absolute ethanol (406 L) was added to provide a mixture having a water content of between 5 and 5.4%. The mixture was then cooled to about 47° C., acetone (975 L) was added and the mixture was stirred while maintaining the temperature. After crystallization, the mixture was stirred for at least one hour, after which an equal volume of acetone was added. The mixture was then slowly cooled to a temperature of about 15° C. and stirred for at least 5 hours. The crystals were collected on a filter and washed with acetone (146 L), then dried under vacuum at NMT 70° C. until the acetone and ethanol levels were less than 1000 ppm and 500 ppm respectively.

### Example 5

### Formation of Form B

Efaproxiral sodium Form A (5.572 kg, 15.33 mole) was added to a solution of acetone (20.3 L) and water (2.0 L), and mixture was heated to dissolution and then cooled to approximately 25° C. The mixture was filtered, and the filtrate was cooled to 18° C., whereupon a precipitate formed. Acetone was added to aid stirring. The mixture was filtered, the filter cake was washed with cold acetone and heptane, and then the filter cake was dried in a vacuum oven (50° C.) for 48 hr. The yield was 4.075 kg (73.1%).

### Example 6

### Single Crystal Structure Determination of Form A Efaproxiral Sodium

Sample Preparation

Crystals of Form A efaproxiral sodium were obtained by an ethanol/methyl t-butyl ether (MTBE) vapor diffusion experiment

Data Collection

A colorless needle of C₂₀H₂₂NO₄Na having approximate dimensions 0.44×0.25×0.15 mm, was mounted on a glass fiber in random orientation. Preliminary examination and data collection were performed with Mo K_{α} radiation (λ=0.71073 Å) on a Nonius KappaCCD diffractometer. Refinements were performed on an LINUX PC using SHELX97 (Sheldrick, G. M. SHELX97, A Program for Crystal Structure Refinement, University of Gottingen, Germany, 1997). The crystallographic drawings were obtained using the programs ORTEP (Johnson, C. K. ORTEPIII, Report ORNL-6895, Oak Ridge National Laboratory, TN, U.S.A. 1996. OPTEP-3 for Windows V1.05, Farrugia, L. J., J. Appl. Cryst. 1997, 30, 565), CAMERON (Watkin, D. J.; Prout, C. K.; Pearce, L. J. CAMERON, Chemical Crystallography Laboratory, University of Oxford, Oxford, 1996), and Mercury (Bruno, I. J. Cole, J. C. Edgington, P. R. Kessler, M. K. Macrae, C. F. McCabe, P. Pearson, J. and Taylor, R. Acta Crystallogr., 2002 B58, 389).

Cell constants and an orientation matrix for data collection were obtained from least-squares refinement using the setting angles of 39725 reflections in the range 2°<θ<25°. The refined mosaicity from DENZO/SCALEPACK (Otwinowski, Z.; Minor, W. Methods Enzymol. 1997, 276, 307.) was 0.37° indicating good crystal quality. The space group was determined by the program ABSEN (McArdle, P. C. J. Appl. Cryst. 1996, 29, 306). There were no systematic absences; the space group was determined to be P 1̅ (no. 2).

The data were collected to a maximum 2θ value of 50.04°, at a temperature of 150±1 K.

Data Reduction

A total of 39725 reflections were collected, of which 20104 were unique. Frames were integrated with DENZO-SMN (Otwinowski, Z.; Minor, W. Methods Enzymol. 1997, 276, 307). Lorentz and polarization corrections were applied to the data. The linear absorption coefficient is 0.99 cm⁻¹ for Mo K_{α} radiation. An empirical absorption correction using SCALEPACK (Otwinowski, Z.; Minor, W. Methods Enzymol. 1997, 276, 307.) was applied. Transmission coefficients ranged from 0.949 to 0.985. Intensities of equivalent reflections were averaged. The agreement factor for the averaging was 7.7% based on intensity.

Structure Solution and Refinement

The structure was solved by direct methods using SIR2002 (Burla, M. C.; Camalli M.; Carrozzini B.; Cascarano G. L.; Giacovazzo C.;. Polidori G.; Spagna, R. J. Appl. Cryst., 2003, 36, 1103). The remaining atoms were located in succeeding difference Fourier syntheses. Hydrogen atoms were included in the refinement but restrained to fide on the atom to which they are bonded. The structure was refined in full-matrix least-squares by minimizing the function:
Σw(|Fₒ|²−|F_{c}|²)²

The weight w is defined as 1/[σ²(Fₒ²)+(0.0337P)²+(0.0000P)], where P=(Fₒ²+2F_{c}²)/3.

Scattering factors were taken from the “International Tables for Crystallography” (International Tables for Crystallography, Vol. C, Kluwer Academic Publishers: Dordrecht, The Netherlands, 1992, Tables 4.2.6.8 and 6.1.1.4). Of the 17939 reflections used in the refinements, only the reflections with Fₒ²>2σ(Fₒ²) were used in calculating R. A total of 10814 reflections were used in the calculation. The final cycle of refinement included 1453 variable parameters and converged (largest parameter shift was 0.002 times its estimated standard deviation) with unweighted and weighted agreement factors of:

$$R = Σ \vert {F}_{o} - {F}_{c} \vert / Σ {F}_{o} = 0.047$$

$${R}_{w} = \sqrt{( Σ {w ( {F}_{o}^{2} - {F}_{c}^{2} )}^{2} / Σ {w ( {F}_{o}^{2} )}^{2} )} = 0.090$$

The standard deviation of an observation of unit weight was 0.919. The highest peak in the final difference Fourier had a height of 0.20 e/Å³. The minimum negative peak had a height of −0.22 e/Å³.

Calculated X-ray Powder Diffraction (XRPD) Pattern

A calculated XRPD pattern was generated for Cu radiation using PowderCell 2.3 and the atomic coordinates, space group, and unit cell parameters from the single crystal data.

Packing Diagrams

Packing diagrams were prepared using CAMERON (Watkin, D. J.; Prout, C. K.; Pearce, L. J. CAMERON, Chemical Crystallography Laboratory, University of Oxford, Oxford, 1996) modeling software. Additional figures were generated using Mercury 1.2 modeling software.

### Example 7

### Conversion of Form A to Form I

Approximately 100 mg of efaproxiral sodium Form A was placed in a 20 mL glass vial. The vial was placed, uncapped, in an ambient temperature relative humidity jar at 75% relative humidity for approximately 24 hours.

### Example 8

### Moisture Balance Experiments for Efaproxiral sodium Form A

A quantity of Form A was placed on a weigh pan at about 0% relative humidity (RH). The RH was slowly raised to about 95% (adsorption) and was then lowered back to about 5% RH (desorption). The weight of the solid material was measured throughout. The results are depicted in FIG. 10. The results suggest that Form A is converted to the tetrahydrate Form I (gaining about 4 moles of water/mole efaproxiral sodium) as the RH is raised to about 85% RH, then to the heptahydrate Form J (gaining about a further 3 moles of water/mole efaproxiral sodium) as the humidity is raised further to about 95% RH. As the humidity is then lowered, the solid material appears to lose about 3 moles of water/mole efaproxiral sodium as the humidity is lowered to about 20% RH, suggesting that Form J converts into Form I as the water is removed from the crystal lattice. The resulting Form I loses slightly less than 4 moles of water as the humidity is further lowered to about 5% RH. The resulting solid material is amorphous efaproxiral sodium (see also EXAMPLE 16).

### Example 9

### Conversion of Form A to Form J

Approximately 100 mg of efaproxiral sodium Form A was placed in a mortar and pestle. Water was added (40 μL) and the material was ground for approximately 30 seconds.

### Example 10

### Conversion of Form I to Form C

Approximately 75 mg of Form J efaproxiral sodium was placed in a 20 mL glass vial. The vial was placed, uncapped, in an 80° C. oven for approximately 3 hours. The oven was at ambient pressure (no vacuum).

### Example 11

### Conversion of Form J to Form C

Approximately 75 mg of efaproxiral sodium Form J was placed in a 20 mL glass vial. The vial was placed, uncapped, in an 80° C. oven for approximately 3 hours at ambient pressure.

### Example 12

### Formation of Form G and Form Q During Recrystallization from Form A in Ethanol and Acetone

Efaproxiral sodium Form A (1125.7 mg) was dissolved completely in 3250 L of ethanol at 48° C. Acetone (7600 μL) was slowly added over a one minute period. Form G and Form Q formed in the reactor at this time. After 1 minute 50 seconds, a further 6250 μL of acetone was added and the reactor was allowed to cool to approximately 25° C. and then to approximately 15° C. The solid material in the reactor during this interval was Form Q. After approximately 10 minutes had passed from the initial addition of acetone, the resulting powdery white solid was collected by vacuum filtration, washed with 6000 μL acetone and dried in a 52° C. vacuum oven. The dried solid was found to be Form Q after 14 hours of drying and also after 32 hours of drying.

### Example 13

### Conversion of Form A to Form G

A slurry of Form A efaproxiral sodium in 9:1 acetonitrile/ethanol was prepared. The slurry material was found to comprise Form G.

### Example 14

### Recrystallization from Methanol to make Form F

A concentrated solution of efaproxiral sodium was prepared in methanol (not saturated, exact concentration is unknown). An aliquot of solution (0.5 mL) was placed in a one-dram glass vial. This one-dram vial was then placed, uncapped, inside a larger 20-mL glass vial containing 4 mL of methyl tertiary-butyl ether (MTBE) (vapor diffusion). The larger vial was capped and the sample was left at ambient to crystallize. Typical sample morphology was observed as needles or fibers (Form F).

### Example 15

### Single Crystal Structure Determination of Form F efaproxiral sodium

Sample Preparation

Crystals of Form F efaproxiral sodium were obtained from a methanol/acetonitrile slurry of efaproxiral sodium.

Data Collection

A colorless plate of C₂₁H₂₆NNaO₅ having approximate dimensions of 0.38×0.35×0.11 mm, was mounted on a glass fiber in random orientation. Preliminary examination and data collection were performed with Mo K_{α} radiation (λ=0.71073 Å) on a Nonius KappaCCD diffractometer. Refinements were performed on an LINUX PC using SHELX97 (Sheldrick, G. M. SHELX97, A Program for Crystal Structure Refinement, University of Gottingen, Germany, 1997). The crystallographic drawings were obtained using the programs ORTEP (Johnson, C. K. ORTEPIII, Report ORNL-6895, Oak Ridge National Laboratory, TN, U.S.A. 1996. OPTEP-3 for Windows V1.05, Farrugia, L. J., J. Appl. Cryst. 1997, 30, 565), CAMERON (Watkin, D. J.; Prout, C K.; Pearce, L. J. CAMERON, Chemical Crystallography Laboratory, University of Oxford, Oxford, 1996), and Mercury (Bruno, I. J. Cole, J. C. Edgington, P. R. Kessler, M. K. Macrae, C. F. McCabe, P. Pearson, J. and Taylor, R. Acta Crystallogr., 2002 B58, 389).

Cell constants and an orientation matrix for data collection were obtained from least-squares refinement using the setting angles of 50155 reflections in the range 2°<θ<23°. The refined mosaicity from DENZO/SCALEPACK (Otwinowski, Z.; Minor, W. Methods Enzymol. 1997, 276, 307.) was 2.95° indicating very poor crystal quality. The space group was determined by the program ABSEN (McArdle, P. C. J. Appl. Cryst. 1996, 29, 306). There were no systematic absences; the space group was determined to be P 1̅ (no. 2).

The data were collected to a maximum 2θ value of 47.6°, at a temperature of 150±1 K.

Data Reduction

A total of 50155 reflections were collected, of which 18687 were unique. Frames were integrated with DENZO-SMN (Otwinowski, Z.; Minor, W. Methods Enzymol. 1997, 276, 307). Lorentz and polarization corrections were applied to the data. The linear absorption coefficient is 0.99 cm⁻¹ for Mo K_{α} radiation. An empirical absorption correction using SCALEPACK (Otwinowski, Z.; Minor, W. Methods Enzymol. 1997, 276, 307.) was applied. Transmission coefficients ranged from unknown minimum to 0.99. Intensities of equivalent reflections were averaged. The agreement factor for the averaging was 13.6% based on intensity.

Structure Solution and Refinement

The structure was solved by direct methods using SIR2002 (Burla, M. C.; Camalli M.; Carrozzini B.; Cascarano G. L.; Giacovazzo C.;. Polidori G.; Spagna, R. J. Appl. Cryst., 2003, 36, 1103). The remaining atoms were located in succeeding difference Fourier syntheses. Hydrogen atoms were included in the refinement but restrained to ride on the atom to which they are bonded. The structure was refined in full-matrix least-squares by minimizing the function:
Σw(|Fₒ|²−|F_{c}|²)²

The weight w is defined as 1/[σ²(Fₒ²)+(0.0651P)²+(2.9980P)], where P=(Fₒ²+2F_{c}²)/3.

Scattering factors were taken from the “International Tables for Crystallography” (International Tables for Crystallography, Vol. C, Kluwer Academic Publishers: Dordrecht, The Netherlands, 1992, Tables 4.2.6.8 and 6.1.1.4). Of the 13421 reflections used in the refinements, only the reflections with Fₒ²>2σFₒ²) were used in calculating R. A total of 7593 reflections were used in the calculation. The final cycle of refinement included 1592 variable parameters and converged (largest parameter shift was 0.004 times its estimated standard deviation) with unweighted and weighted agreement factors of:

$$R = Σ \vert {F}_{o} - {F}_{c} \vert / Σ {F}_{o} = 0.073$$

$${R}_{w} = \sqrt{( Σ {w ( {F}_{o}^{2} - {F}_{c}^{2} )}^{2} / Σ {w ( {F}_{o}^{2} )}^{2} )} = 0.151$$

The standard deviation of an observation of unit weight was 1.03. The highest peak in the final difference Fourier had a height of 0.20 e/Å³. The minimum negative peak had a height of −0.24 e/Å³.

Calculated X-ray Powder Diffraction (XRPD) Pattern

A calculated XRPD pattern was generated for Cu radiation using PowderCell 2.3 and the atomic coordinates, space group, and unit cell parameters from the single crystal data.

Packing Diagrams

Packing diagrams were prepared using CAMERON (Watkin, D. J.; Prout, C. K.; Pearce, L. J. CAMERON, Chemical Crystallography Laboratory, University of Oxford, Oxford, 1996) modeling software. Additional figures were generated using Mercury 1.2 modeling software.

### Example 16

### Amorphous Efaproxiral Sodium

Amorphous efaproxiral sodium was obtained by freeze-drying efaproxiral sodium dissolved in 5:1 dioxane/water.

### Example 17

### Formulation of Efaproxiral Sodium

A sample of efaproxiral sodium prepared as described in EXAMPLE 1 and 4 was formulated for use as a drug product as follows: To a 1 L volumetric flask was added sodium chloride (2.25 g), anhydrous monobasic sodium phosphate (135 mg) and dibasic sodium phosphate, heptahydrate (7 mg), followed by approximately 800 mL of deionized water. The mixture was mixed until all of the solids had dissolved. To this solution was added efaproxiral sodium (21.3 g). The mixture was again mixed until all of the solids had dissolved. The pH of the resulting solution was then adjusted to approximately 7.9 using 0.1N HCl. Finally, the solution was diluted to volume using deionized water. The resulting solution represents a formulated efaproxiral sodium drug product. A sample of the formulated efaproxiral sodium drug product (50 mL) prepared, was placed into a 50 mL glass syringe. To the syringe was attached one of three 0.22 μm, 25 mm disposable syringe filters (3.9 cm² filter area). The solution was then pushed through the selected filter at a rate of approximately 8 mL/min. The entire 50 mL of filtrate was collected in a clean glass container.

1. Unsolvated crystalline efaproxiral sodium having an X-ray powder diffraction pattern with at least one peak selected from the group consisting of 3.2±0.2° and 9.7±0.2° in 2θ when obtained from a copper radiation source (CuKα λ=1.54 Å).

2. The unsolvated crystalline efaproxiral sodium of claim 1 which is further characterized by an X-ray powder diffraction pattern with at least one further peak selected from the group consisting of 7.6±0.2°, 8.2±0.2°, 12.9±0.2°, 15.3±0.2°, 16.4±0.2°, 17.4±0.2°, and 18.5±0.2° in 2θ when obtained from a copper radiation source (CuKα λ=1.54 Å).

3. The unsolvated crystalline efaproxiral sodium of claim 1 which is further characterized by a reflectance Fourier transform infrared (FTIR) spectrum which shows at least one absorption band selected from the group consisting of 3274±2 cm⁻¹, 955±2 cm⁻¹ and 736±2 cm⁻¹.

4. Unsolvated crystalline efaproxiral sodium having an X-ray powder diffraction pattern with at least one peak selected from the group consisting of 11.5±0.2°, 14.0±0.2°, and 19.4±0.2° in 2θ when obtained from a copper radiation source (CuKα λ=1.54 Å).

5. The unsolvated crystalline efaproxiral sodium of claim 4 which is further characterized by an X-ray powder diffraction pattern with at least one further peak selected from the group consisting of 8.6±0.2°, 15.1±0.2°, 16.5±0.2°, 18.0±0.2°, and 20.6±0.2° in 2θ when obtained from a copper radiation source (CuKα λ=1.54 Å).

6. The unsolvated crystalline efaproxiral sodium of claim 4 which is further characterized by reflectance Fourier transform infrared (FTIR) spectrum which shows at least one absorption band selected from the group consisting of 3289±2 cm⁻¹, 1338±2 cm¹, and 730±2 cm⁻¹.

7. A crystalline solvate comprising efaproxiral sodium and a solvent selected from group consisting of water, ethanol, methanol and acetone.

8. The crystalline solvate of claim 7 wherein the solvent is water.

9. The crystalline solvate of claim 8 which comprises less than four moles of water per mole of efaproxiral sodium.

10. The crystalline efaproxiral sodium hydrate of claim 8 which is characterized by an X-ray powder diffraction pattern with a peak at 7.7±0.2° in 2θ when obtained from a copper radiation source (CuKα λ=1.54 Å).

11. The crystalline efaproxiral sodium hydrate of claim 10 which is further characterized by an X-ray powder diffraction pattern with at least one further peak selected from the group consisting of 3.1±0.2°, 9.4±0.2°, 12.5±0.2° and 15.6±0.2° in 2θ when obtained from a copper radiation source (CuKα λ=1.54 Å).

12. The crystalline efaproxiral sodium hydrate of claim 8, which is further characterized by a reflectance Fourier transform infrared (FTIR) spectrum which shows an absorption band 2225±2 cm⁻¹.

13. The crystalline solvate of claim 8 which comprises about four moles of water per mole of efaproxiral sodium.

14. The crystalline efaproxiral sodium hydrate of claim 8 which is characterized by an X-ray powder diffraction pattern with a peak at 11.0±0.2° in 2θ when obtained from a copper radiation source (CuKα λ=1.54 Å).

15. The crystalline efaproxiral sodium hydrate of claim 14 which is further characterized by an X-ray powder diffraction pattern with at least one further peak selected from the group consisting of 2.7±0.2°, 8.2±0.2°, 14.7±0.2°, 15.7±0.2°, and 16.1±0.2° in 2θ when obtained from a copper radiation source (CuKα λ=1.54 Å).

16. The crystalline solvate of claim 8 which comprises about seven moles of water per mole of efaproxiral sodium.

17. The crystalline efaproxiral sodium hydrate of claim 8 which is characterized by an X-ray powder diffraction pattern with a peak at 19.2±0.2° in 2θ when obtained from a copper radiation source (CuKα λ=1.54 Å).

18. The crystalline efaproxiral sodium hydrate of claim 8 which is characterized by an X-ray powder diffraction pattern with peaks at 12.1±0.2° and 12.8±0.2° in 2θ when obtained from a copper radiation source (CuKα λ=1.54 Å).

19. The crystalline efaproxiral sodium hydrate of claim 17 which is further characterized by an X-ray powder diffraction pattern with at least one further peak selected from the group consisting of 3.2±0.2°, 14.7±0.2°, 15.7±0.2°, and 16.1±0.2° in 2θ when obtained from a copper radiation source (CuKα λ=1.54 Å).

20. The crystalline efaproxiral sodium hydrate of claim 8, which is further characterized by a reflectance Fourier transform infrared (FTIR) spectrum which shows at least one absorption band selected from the group consisting of 3618±2 cm⁻¹, 1921±2 cm⁻¹, and 1028±2 cm¹.

21. The crystalline solvate of claim 7 wherein the solvent is ethanol.

22. The crystalline solvate of claim 21 which comprises about one mole of ethanol per mole of efaproxiral sodium.

23. The crystalline efaproxiral sodium solvate of claim 21 which is characterized by an X-ray powder diffraction pattern which lacks peaks at 3.0±0.2° and 3.8±0.2° in 2θ and when obtained from a copper radiation source (CuKα λ=1.54 Å).

24. The crystalline efaproxiral sodium solvate of claim 21 which is characterized by an X-ray powder diffraction pattern with at least one peak selected from the group consisting of 10.2±0.2°, 16.7±0.2°, and 17.6±0.2° in 2θ when obtained from a copper radiation source (CuKα λ=1.54 Å).

25. The crystalline efaproxiral sodium solvate of claim 23 which is further characterized by an X-ray powder diffraction pattern with at least one peak selected from the group consisting of 8.3±0.2°, 8.5±0.2°, 11.6±0.2°, 13.0±0.2°, 14.6±0.2°, 18.1±0.2°, and 20.5±0.2° in 2θ when obtained from a copper radiation source (CuKα λ=1.54 Å).

26. The crystalline efaproxiral sodium solvate of claim 22 which is further characterized by a reflectance Fourier transform infrared (FTIR) spectrum which shows at least one absorption band selected from the group consisting of 3086±2 cm⁻¹, 1088±2 cm⁻¹, and 903±2 cm⁻¹.

27. A crystalline efaproxiral sodium solvate according to claim 21 having an X-ray powder diffraction pattern with at least one peak selected from the group consisting of 3.0±0.2°, 3.8±0.2°, 6.5±0.2°, 9.2±0.2°, and 12.2±0.2° in 2θ when obtained from a copper radiation source (CuKα λ=1.54 Å).

28. Crystalline efaproxiral sodium characterized by an X-ray powder diffraction pattern with peaks at 3.0±0.2° and 3.8±0.2° in 2θ when obtained from a copper radiation source (CuKα λ=1.54 Å).

29. The crystalline efaproxiral sodium according to claim 27 which is further characterized by an X-ray powder diffraction pattern with at least one further peak selected from the group consisting of 7.7±0.2°, 8.4±0.2°, 13.4±0.2°, 15.4±0.2°, 15.8±0.2°, 16.2±0.2°, 18.3±0.2°, 19.3±0.2°, 19.8±0.2°, and 20.1±0.2° in 2θ when obtained from a copper radiation source (CuKα λ=1.54 Å).

30. The crystalline solvate of claim 7 wherein the solvent is acetone.

31. The crystalline solvate of claim 30 which comprises between about 1 mole and about ½ mole of acetone per mole of efaproxiral sodium.

32. The crystalline efaproxiral sodium solvate of claim 30 which is characterized by an X-ray powder diffraction pattern with at least one peak selected from the group consisting of 3.7±0.2°, 6.5±0.2°, 8.4±0.2°, 16.2±0.2°, 18.3±0.2, 19.4±0.2°, 19.8±0.2°, and 20.1±0.2° in 2θ when obtained from a copper radiation source (CuKα λ=1.54 Å).

33. The crystalline efaproxiral sodium solvate of claim 30, which is further characterized by a reflectance Fourier transform infrared (FTIR) spectrum which shows at least one absorption band selected from the group consisting of 3380±2 cm⁻¹, 1701±2 cm¹, and 1645±2 cm¹.

34. The crystalline solvate of claim 7 wherein the solvent is methanol.

35. The crystalline solvate of claim 34 which comprises about 1 mole of methanol per mole of efaproxiral sodium.

36. The crystalline efaproxiral sodium solvate of claim 34 which is characterized by an X-ray powder diffraction pattern with at least one peak selected from the group consisting of 4.3±0.2°, 8.9±0.2°, 9.2±0.2°, 11.5±0.2°, 13.8±0.2°, 14.3±0.2°, 15.8±0.2°, 16.2±0.2°, 17.8±0.2°, and 18.7±0.2° in 2θ when obtained from a copper radiation source (CuKα λ=1.54 Å).

37. The crystalline efaproxiral sodium solvate of claim 34, which is further characterized by a reflectance Fourier transform infrared (FTIR) spectrum which shows at least one absorption band selected from the group consisting of 747±2 cm⁻¹, 1053±2 cm⁻¹, 1338±2 cm⁻¹, and 1562±2 cm⁻¹.

38. Amorphous efaproxiral sodium.

39. A pharmaceutical formulation comprising the crystalline efaproxiral sodium form of claim 1 and one or more pharmaceutical carriers, diluents, or excipients.

40. A method for the preparation of an aqueous solution of efaproxiral sodium, the method comprising dissolving solid efaproxiral sodium of claim 1 in a solution comprising water.

41. An aqueous solution of efaproxiral sodium produced according to the method of claim 40.

42. A method for preparing Form A crystalline efaproxiral sodium, the method comprising:
(a) dissolving efaproxiral sodium in water to form an aqueous solution;
(b) concentrating said aqueous solution to remove the maximum amount of water while maintaining the aqueous solution at a temperature of about 50° C.;
(c) adding ethanol to the concentrated aqueous solution to provide a mixture having less than about 15 weight percent water content;
(d) cooling the ethanol/water mixture of (c) without precipitating the efaproxiral sodium from the ethanol/water mixture;
(e) adding acetone to the ethanol/water mixture to precipitate crystalline efaproxiral sodium; and
(f) cooling the mixture of step (d) to below about 25° C. with stirring.

43. A method for preparing Form B crystalline efaproxiral sodium, the method comprising:
(a) dissolving efaproxiral sodium in acetone and water with heating to form a solution; and
(b) cooling said solution to precipitate efaproxiral sodium crystals.

44. A method for preparing Form I crystalline efaproxiral sodium comprising incubating Form A crystalline efaproxiral sodium at high relative humidity.

45. A method for preparing Form J crystalline efaproxiral sodium comprising slurrying Form A crystalline efaproxiral sodium in water.

46. A method for preparing Form C crystalline efaproxiral sodium comprising dehydrating Form I crystalline efaproxiral sodium or Form J crystalline efaproxiral sodium.

47. A method for preparing Form Q crystalline efaproxiral sodium comprising:
(a) dissolving efaproxiral sodium in ethanol with stirring at elevated temperature.
(b) adding acetone to the solution of (a) with continued stirring;
(c) cooling the solution of (b) below 25° C. with continued stirring wherein crystalline efaproxiral sodium is formed.

48. A method for producing Form G crystalline efaproxiral sodium comprising slurrying Form A crystalline efaproxiral sodium in a mixture of acetonitrile and ethanol.

49. A method for producing Form F crystalline efaproxiral sodium comprising dissolving efaproxiral sodium in methanol and removing said methanol using vapor diffusion.

50. A method for producing Form P crystalline efaproxiral sodium comprising dissolving efaproxiral sodium in ethanol and cooling said solution to precipitate crystalline efaproxiral sodium.

51. Crystalline efaproxiral sodium produced according to the method of any one of claims 42-50.

52. A method for producing amorphous efaproxiral sodium comprising freeze-drying efaproxiral sodium dissolved in dioxane and water.

53. A method for treating a condition selected from the group consisting of whole body or tissue hypothermia, hypoxia or hypotension, wounds, brain injury, diabetic ulcers, chronic leg ulcers, pressure sores, tissue transplants, stroke or cerebro ischemia, ischemia or oxygen deprivation, respiratory disorders including acute respiratory distress syndrome and chronic obstructive pulmonary disorder, surgical blood loss, sepsis, multi-system organ failure, normovolemic hemodilution procedures, carbon monoxide poisoning, bypass surgery, carcinogenic tumors, and oxygen deprivation of a fetus comprising the step of administering to a patient suffering from or undergoing said condition a sufficient quantity of the composition of any one of claims 1, 4, 7, 28, 38, 41, 57, 60, 63 and 66.

54. The crystalline efaproxiral sodium according to claim 28 which is further characterized by an X-ray powder diffraction pattern with at least one further peak selected from the group consisting of 7.7±0.2°, 8.4±0.2°, 13.4±0.2°, 15.4±0.2°, 15.8±0.2°, 16.2±0.2°, 18.3±0.2°, 19.3±0.2°, 19.8±0.2° and 20.1±0.2° in 2θ when obtained from a copper radiation source (CuKα λ=1.54 Å).

55. A pharmaceutical formulation comprising the crystalline efaproxiral sodium form of claim 4 and one or more pharmaceutical carriers, diluents, or excipients.

56. A method for the preparation of an aqueous solution of efaproxiral sodium, the method comprising dissolving solid efaproxiral sodium of claim 4 in a solution comprising water.

57. An aqueous solution of efaproxiral sodium produced according to the method of claim 56.

58. A pharmaceutical formulation comprising the crystalline efaproxiral sodium form of claim 7 and one or more pharmaceutical carriers, diluents, or excipients.

59. A method for the preparation of an aqueous solution of efaproxiral sodium, the method comprising dissolving solid efaproxiral sodium of claim 7 in a solution comprising water.

60. An aqueous solution of efaproxiral sodium produced according to the method of claim 59.

61. A pharmaceutical formulation comprising the crystalline efaproxiral sodium form of claim 28 and one or more pharmaceutical carriers, diluents, or excipients.

62. A method for the preparation of an aqueous solution of efaproxiral sodium, the method comprising dissolving solid efaproxiral sodium of claim 28 in a solution comprising water.

63. An aqueous solution of efaproxiral sodium produced according to the method of claim 62.

64. A pharmaceutical formulation comprising the amorphous efaproxiral sodium form of claim 38 and one or more pharmaceutical carriers, diluents, or excipients.

65. A method for the preparation of an aqueous solution of efaproxiral sodium, the method comprising dissolving solid efaproxiral sodium of claim 38 in a solution comprising water.

66. An aqueous solution of efaproxiral sodium produced according to the method of claim 65.

67. A method for treating a condition selected from the group consisting of whole body or tissue hypothermia, hypoxia or hypotension, wounds, brain injury, diabetic ulcers, chronic leg ulcers, pressure sores, tissue transplants, stroke or cerebro ischemia, ischemia or oxygen deprivation, respiratory disorders including acute respiratory distress syndrome and chronic obstructive pulmonary disorder, surgical blood loss, sepsis, multi-system organ failure, normovolemic hemodilution procedures, carbon monoxide poisoning, bypass surgery, carcinogenic tumors, and oxygen deprivation of a fetus comprising the step of administering to a patient suffering from or undergoing said condition a sufficient quantity of a composition selected from claim 51.
